(19) United States
(12) Patent Application Publication
(10) Pub. No.: US 2003/0181362 A1
(43) Pub. Date: Sep. 25, 2003

(54) Receptor

(76) Inventor: Jameel Inal, Basel (CH)

Correspondence Address: Nixon & Vanderhye, 8th Floor, 1100 North Glebe Road, Arlington, VA, 22201-4714, US

(21) Appl. No.: 10/169,710

(22) PCT Filed: Jan. 10, 2001
(86) PCT No.: PCT/GB01/00085

(30) Foreign Application Priority Data
Jan. 10, 2000 (GB) 0000474.7

(51) Int. Cl. A61K 38/12

(52) U.S. Cl. 514/9

(57) ABSTRACT

The invention relates to peptides in the TORE protein family, and use of such peptides in medicine.

The present invention relates to human and parasite orphan receptor proteins.

Schistosomes are the causative agents of schistosomiasis, an important and widespread disease in tropical regions and Trypanosoma cruzi is a protozoan parasite that causes Chagas' disease in humans. Surface proteins of schistosomes, particularly in the adult worm and larval or schistosomula stages as well as surface molecules of the infective trypomastigote stage of trypanosomes, represent potential vaccine candidates because of their accessibility to immune effector mechanisms of the host.

In schistosomes, some surface proteins, such as the 23 kDa group of surface antigens [Wright, M. D. et al. (1990) J. Immunol. 144, 3195-3200, Davern, K. M. et al. (1991) Mol. Biochem. Parasitol. 48, 67-76 and Inal, J. and Bickle, Q. (1995) Mol. Biochem. Parastiol. 74, 217-221], show significant homology at the amino acid level, and in the resulting membrane topology with various mammalian leukocyte cell surface antigens. The conservation of the 23 kDa parasite surface molecules with these leukocyte cell surface molecules (‘molecular mimicry’) suggests that they are of fundamental biological importance.

Such examples of molecular mimicry may have the function of disguising the parasite as ‘self’ in terms of the host immune system, and help the parasite avoid the host immune response. Therefore, an understanding of such proteins is useful in developing strategies to modulate the human immune response. For example, parasite surface proteins could bind to and remove a component of the immune response directed against the parasite. In this way the parasite receptor plays a directly protective role.

Therefore, the identification of parasite surface proteins, especially those with human homologues, is potentially valuable in the development of vaccines, and additionally in the understanding and modulation of the human immune response. However, targeted application of such proteins or peptides to therapy depends upon the identification of the appropriate host responses to the protein or peptide.

Recently, two transmembrane proteins have been identified in the blood-dwelling human schistosome parasites, Schistosoma haematobium and Schistosoma mansoni, designated ShTORE (Schistosome haematobium Trispanning Orphan Receptor) and SmTORE (Schistosome mansoni Trispanning Orphan Receptor) respectively {Inal, Biochimica et Biophysica Acta, 1445 (1999) 283-298}. It has been suggested that the surface location of the ShTORE protein makes it a potential vaccine candidate for schistosomiasis. However, nothing more is known about the possible role of the TORE proteins in the human or other hosts, or those elements of the TORE proteins which are biologically important.

Accordingly, there is still a need to further understand the possible role of the TORE proteins in humans and other hosts.

The present invention sets out to address this need.

In a first aspect, the present invention relates to the use of a peptide comprising the sequence of SEQ ID NO 1 or SEQ ID NO 2, or fragment thereof, or comprising a mutant or variant of SEQ ID NO 1, SEQ ID NO 2 or fragment thereof, in medicine.

In a further aspect, the present invention relates to the use of a peptide comprising the sequence of SEQ ID NO 1 or SEQ ID NO 2, or fragment thereof, or comprising a mutant or variant of SEQ ID NO 1, SEQ ID NO 2 or fragment thereof, in the preparation of a medicament for modulation of complement activity.

We have now surprisingly discovered that the ShTORE protein is capable of binding the C2 protein component of complement and, thereby, inhibiting the complement reaction. Interaction between C2 and TORE is thought to occur through the putative extracellular domains (herein “ed”) of the TORE protein, which correspond to SEQ ID NO 1 (MSPSLVSDTQKHERGFHEVKIKHFSPY) and SEQ ID NO 2 (SSTSDIRLVIHTKTGPIYIKST). For the schistosomes, inhibition of complement has the advantage of avoiding the host immune response. However, in therapeutic terms, this finding indicates the potential for use in the present invention of the TORE protein and protein fragments, along with mutants and variants thereof, in the inhibition of complement action, thereby acting as anti-inflammatory agents, for example. Accordingly the invention preferably relates to proteins from any TORE family, or peptides derived from such proteins, or homologues of such proteins or peptides, which are active in the inhibition of complement activation, and to the use of such proteins and peptides in medicine.

The term ‘TORE’, as used herein, refers to any protein or peptide which is a member of the TORE family of proteins by virtue of sequence or functional homology with any of the three sequenced TORE proteins listed herein. Preferably family members have at least 20%, preferably 30% or more homology at the amino acid level over the full length TORE sequence, more preferably 40%, 50% or even higher. Preferably a TORE protein comprises one or both of SEQ ID NO 1 or 2, or sequences homologous thereto, which correspond to the putative extracellular domains of the protein. Preferably, family members have 3, 5 or 7 transmembrane domains, and may be identified by biological interaction, for example, as being proteins or peptides capable of reacting with antibodies generated to TORE proteins or peptides, for example antibodies to SEQ ID NO 1 or 2.

In view of the biological activity of the TORE extracellular domain 1 (ed-1) peptide, the Trispanning Orphan Receptor (TORE) family of proteins is also referred to herein as the “Complement Receptor Inhibitory Trispanning” (CRIT) family of proteins, and the term ‘TORE’ is used interchangeably with the term ‘CRIT’ herein.

Preferred regions of TORE which serve to modulate complement action are SEQ ID NO 1 and 2, which correspond to 2 putative extracellular domains of ShTORE, shown to inhibit complement action in vitro. Accordingly, these peptides are preferred for use in the present invention.

The invention also relates to peptides which comprise fragments of SEQ ID NO 1 or 2. Preferred peptides of the invention are short, for example, less than 20 amino acids, preferably less than 15 amino acids, more preferably 11 amino acids or even less.

Suitably, peptide fragments are functionally equivalent to SEQ ID NO 1 or 2, in that they are able to inhibit complement activation, as assessed for example, by in vitro experiments outlined herein. Preferably, the peptide fragments have substantially similar activity in complement inhibition. However, peptides for use in the invention may even possess a lower degree of complement inhibitory activity where this activity is sufficient for the peptide application, for example, in therapeutic treatment.

The invention also relates to peptides comprising mutants or variants of SEQ ID NO 1 and 2 or peptide fragments thereof, for use in the present invention. Suitably the mutant or variant is functionally equivalent to SEQ ID NO 1 and 2, as defined above. Essentially any mutant or variant type, such as a deletion, substitution, inversion or addition mutant of the peptide or peptide fragment, or a variant such as a derivative, whether naturally occurring or synthetic, is contemplated.

Preferably the peptides comprising mutants and variants of SEQ ID NO 1 or 2 or peptide fragments thereof are at least 50% as effective as those of SEQ ID NO 1 and 2 in the inhibition of complement, more preferably 60%, 70%, 80%, 90% or higher. Preferably peptide fragments, mutants or variants are more effective than SEQ ID NO 1 and 2 in the inhibition of complement. Inhibition of complement may be assessed using in vitro techniques herein described.

For peptides comprising fragments, mutants or variants of SEQ ID NO 1 or 2, we prefer that the fragment mutant or variant has at least 30% amino acid identity over the region of SEQ ID NO 1 or 2, more preferably 35%, preferably 40%, 50%, 55% or even higher. Suitably the peptides also have at least 50% similarity, preferably 60%, more preferably 65%, most preferably 70% similanrty or even higher over the region of SEQ ID NO 1 or 2, or part thereof.

Suitably, homology of peptide fragments may be assesses using the default settings for search facilities at http://www.expasy.ch/tools/scnpsit2.html. For example, the invention relates to peptides identifiable by use of the scanprosite program for searching the SwissProt and TrEMBL databases with the protein motif EVKIX4PY. Use of the blastp program under default settings, or by varying standard parameters with the nr (non redundant) database, for example can also be used to identify homologous sequences, as can the use of the TBLASTN program.

Preferably peptides for use in the present invention consist of SEQ ID NO 1 or 2, or are fragments, mutants or variants thereof.

Preferred for use in the present invention are peptide regions derived from the TORE family of proteins, which are predicted or known to adopt an extracellular location. The present invention also relates to fragments, mutants or variants of such peptides, as defined above. Putative extracellular location can be predicted using techniques described herein.

Particularly preferred are peptides which are able to interact with human C2 complement protein, to inhibit complement activation. In vitro techniques to assess complement activation via cell lysis are described herein, and can be used to identify suitable peptides.

In a further aspect of the invention, it is known that human C2 is known to interact with the complement protein C4b. Comparison of the sequences of C4b and ShTORE indicates, surprisingly, a significant homology between the first extracellular domain of the TORE proteins and C4b β chain. This allows a consensus sequence to be derived for peptides which interact with the C2 protein in this way. The consensus sequence is EVKI-Xn-PY, where “X” indicates any amino acid, and “n” indicates a spacer of between 1-6 amino acids between the conserved EVKI and PY elements, most preferably 4 amino acids. The extracellular domain with which C4b β chain is homologous is the predicted first (N-terminal) extracellular domain of ShTORE. The first extracellular domain of any TORE protein is herein referred to as “ed-1”.

Therefore, in a further aspect, the present invention relates to the use of a peptide comprising the amino acid sequence EVKI-X(n)-PY in medicine, wherein “X” is any amino acid, and “n” is 1-6.

In a yet further aspect, the invention relates to use of a peptide comprising the amino acid sequence EVKI-X(n)-PY in the preparation of a medicament for modulation of human complement activity, preferably inhibition of human complement protein C2 activity, wherein “X” is any amino acid, and “n” is 1-6.

In the present study, a 27-mer peptide identical to the ed-1 of ShTORE is shown herein to interact with human complement protein C2 (Example 1.6). The interaction of ShTORE extracellular domain 1 with C2 is shown herein to prevent the normal interaction of C2 with other complement proteins, and thereby to inhibit complement activation (Example 1.7). As such, we prefer that the peptides of the present invention comprising the EVKI-X(n)-PY motif are able to interact with human C2 complement protein, and to functionally inhibit complement activation.

Most preferred are peptides MSPSLVSHTQKNERGSHEVKIEHFTPY, MSPSLVSTYQKNERGSHEVKIKHFSPY and MSPSLVSDTQKHERGSHEVKIKHFSPY, which represent the ed-1 sequences of the Rat TORE, SmTORE and ShTORE proteins. The invention also relates to mutants and variants of these peptides in which one or more amino acids have been inserted, deleted or substituted without affecting the biological activity of the peptide in interaction with C2.

The 4 amino acids between the ‘EVKI’ and the ‘PY’ motifs are variable, and the number of amino acids may also be varied, with the constraint that the peptide is still able to interact with the C2 protein. For example, X may be suitably from 1-6 amino acids, or more, provided that biological activity of the peptide on complement activity modulation is not significantly affected. Therefore, the term “EVKI-X(n)-PY”, as used herein, includes the possibility of X being between 1-6 amino acids, with the above functional constraint. However, X=3-5 is preferred, with X=4 most preferred.

Within the human immune system, the C2 protein normally binds with the C4b protein to form the so-called ‘Classical C3 convertase’. Analysis of the extracellular domain 1 of the rat and schistosome sequences indicates that there are two areas of homology between extracellular domain 1 and the C4b sequence. The sequence similarity between C4b and the ed-1 sequences indicates that the extracellular domain 1 peptide may compete with C4b to bind to the C2 complement protein. In this way, the extracellular domain 1 peptide may be used to modulate the immune response. Therefore, we further prefer that the peptide of the present invention is able to compete with C4b for the binding of C2. Competition for binding may be assessed by competition binding assays, for example, which are well known in the art.

C2 has been shown to preferentially interact with the dimeric form of ShTORE. The dimeric form of the ShTORE protein is also relevant to protein phosphorylation. Specifically, ShTORE is found to be phosphorylated on tyrosine residues primarily in the dimeric form, although there is a low level of phosphorylation in the monomeric and trimeric forms. Therefore we prefer the peptide of the invention is a dimer. We further prefer that the peptide of the present invention is able to form covalent oligomers, such as dimers and trimers. We also prefer that the protein of the present invention interacts with C2 in a dimeric form.

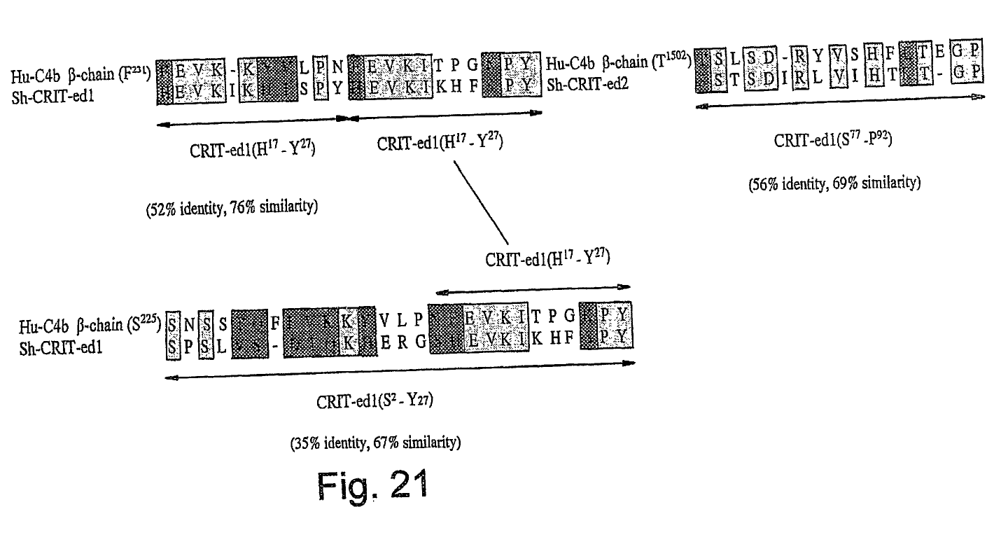

Preferably a dimeric peptide is simply a linear direct repeat of a TORE peptide, for example HEVKIKHFSPYHEVKIKHFSPY, as shown in FIG. 21. Optionally, direct repeats may comprise a spacer region between the direct repeat sequence.

Also preferred are peptides having the general sequence [F/H]-E-V-K-X(0, 1)-X(4)-P-[Y/N][F/H]-E-V-K-X(0,1)-X(4)-P-[Y/N], based upon the alignment of FIG. 21, and generally E-V-K-X(0,8)-E-V-K-I-X(4)-P-Y. Generally preferred are sequences in which the EVK element is repeated with a 7 or 8 amino acid gap. The EVK sequence is spaced in this way in C4b, and the same pattern is seen if two HEVKIKHFSPY sequences are laid contiguously (i.e the C-terminal part of the ed1 peptide), to give (hEVKikhfspyhEVKikhfspy).

Also preferred are cyclic peptides, such as:

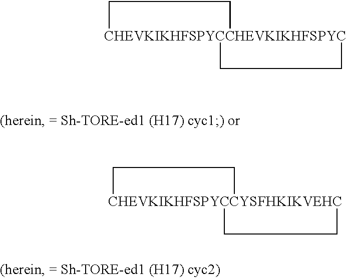

Two sequences present in C4b are homologous to ed-1, as shown in FIG. 14. This observation is consistent with interaction of C2 with a dimeric form of ShTORE. Therefore, in addition to peptides which comprise the EVKI-X(n)-PY motif, the present invention also relates to peptides that comprise an amino acid sequence which is conserved between either of the C4b domain homologous sequences and the known ed-1 sequences, namely SXSX(7)KX(6)EVKIX(4)PY or SX(4)VX(7)RGX(2)EX(7)P, where X represents any amino acid and a numeral represents the number of ‘X’ (unspecified) amino acids. We particularly prefer that the peptide of the present invention has the consensus sequence: Serine-X16-glutamic acid-X7-Proline, which is the consensus sequence derived from the comparison of both C4b sequences, and the TORE ed-1 peptides. Suitably, such peptides may be used in the preparation of a medicament for the inhibition of human complement protein C2 activity, or modulation of complement activity. In a yet further aspect, the invention relates to pharmaceutical compositions comprising such peptides.

The term “interact”, as used herein, refers to the ability of a peptide, for example, to bind to the human C2 protein, preferably to produce a biological effect. Interaction may suitably be assessed by, for example, affinity chromatography (outlined in Example 1.4). Interaction may also be assessed by co-precipitation with the C2 protein using either antibodies to C2 or to the extracellular domain 1. Means to determine co-precipitation or affinity binding are readily apparent to the person skilled in the art. Interaction may also be inferred by indirect effects, for example, by use of competition binding studies to assess peptide inhibition of complement mediated cell lysis.

Preferred peptides of the invention which modulate C2 activity are preferably short peptides which may be used therapeutically and easily delivered. Peptides of less than 100 amino acids in length are preferred, with peptides of less than 50 amino acids more preferable and peptides less than 30 amino acids, preferably less than 15 amino acids most preferred.

In a further embodiment of the invention, the amino acid sequence of the rat homologue of the TORE protein has been determined (herein “RaTORE”), and the human homologue identified. The present invention thus extends to both these proteins and fragments thereof, along with pharmaceutical preparations containing such peptides, and the use of such peptides in the preparation of a composition for inhibition of human complement protein C2 activity, or modulation of complement activity.

Sequencing of the rat protein allows further information to be derived concerning the biologically important amino acids of the ed-1 region. Using the rat sequence in conjunction with the known parasite sequences, a consensus sequence can be derived for the conserved (and, therefore, functionally important regions) of the TORE protein family.

The N-terminal 27 amino acids of the RaTORE, SmTORE and ShTORE proteins, respectively are as follows:

1 MSPSLVSHTQKNERGSHEVKIEHFTPY

2 MSPSLVSTYQKNERGSHEVKIKHFSPY

3 MSPSLVSDTQKHERGSHEVKIKHFSPY

Therefore, the present invention also relates to any peptide having the consensus sequence MSPSLVSXTQKXERGSHEVKIXHFXPY (with the exception of those derived from Schistosoma haematobium and Schistosoma mansoni), and to pharmaceutical preparations containing such peptides. In the above sequence, X represents any amino acid.

The present invention also relates to a protein or peptide which has at least 75% identity with the ed-1 of the RaTORE protein, SmTORE or ShTORE protein. By this, it is meant that any peptide of the invention may be aligned with the RaTORE sequences such that there are at least 21/27 identical amino acids in equivalent relative positions over that region of the peptide sequence. We prefer that the peptides of the present invention have greater than 80% identity with RaTORE extracellular domain 1, preferably greater than 85% identity, with greater than 90% identity particularly preferred.

We particularly prefer that the peptides of the invention contain at least the sequence MSPSLVS or ERGSHEVKI, preferably both, which are conserved between all three sequences in the putative ed-1 domain.

Particularly preferred is the sequence HEVKIKHFSPY, a fragment of SEQ ID NO 1, capable of effective complement inhibition in vitro.

Peptides of the present invention have also been shown to reduce inflammation in vivo. For example, the TOR-ed1 peptide HEVKIKSPY has also been shown to reduce inflammation mediated by immune complexes and Classical pathway complement (Type III hypersensitivity) in vivo and points to its possible use in more specific disease models involving complement such as adjuvant-induced arthritis or animal models for adult respiratory distress syndrome, multiple sclerosis, ischaemia/reperfusion and graft rejection.

In a further aspect, the present invention also relates specifically to the use of peptides derived from human C4b protein in medicine, and to mutants and variants thereof, as defined above, having complement inhibitory activity. Particularly preferred is use of such peptides in the preparation of a medicament for modulation of complement activity, preferably inhibition of complement. In particular we have determined that peptide fragments derived from C4b are able to inhibit complement activity, although full length C4b is required for complement activation. Accordingly fragments of C4b, such as SNSSTQFEVKKYVLPNFEVKITPGKPY shown in FIG. 21 represent variants of SEQ ID NO 1, as defined above.

The sequence of SEQ ID NO 2 represents a second putative extracellular ed-2 sequence of ShTORE, having the sequence SSTSDIRLVIHTKTGPIYIKST. We have also determined that the ed-2 region has homology with the C4b, this time with the C4b γ chain, as illustrated in FIG. 21. Accordingly, the invention also particularly relates to TORE peptides, or other peptides having homology with the γ chain of C4b, preferably with at least 50% identity over the ed-2 region, more preferably 55% identity, or even higher.

Also preferred are peptides derived from the C4b γ chain, for use in the present invention. Analogous to the C4b β chain peptides, fragments of the C4b γ chain may be used to prevent C2- C4b interaction, and inhibit complement.

Most preferred are peptides which have sequence homology at the protein or DNA level with the TORE family of proteins, particularly with the putative extracellular regions ed-1 or ed-2, as assessed by (for example) BLAST or other search tools using standard parameters. Preferably the C4b peptides have at least 30% identity over the ed-1 or ed-2 region, more preferably 35%, 40%, 50% or even higher. Suitably the C4b peptides have at least 50% similarity, preferably 60%, 65%, most preferably 70% similarity or even higher over the ed-1 region or ed-2 region, or part thereof, as assessed using the search tools described herein. Most preferred are peptides derived from C4b which inhibit complement activity. Inhibition of complement may be assessed by in vitro lysis assays as described herein. Most preferred is the peptide C4b β chain derived peptide N′-C′ FEVKITPGKPY, which comprises the preferred ed-1 consensus sequence of EVKI-X(n)-PY, and which has been shown to inhibit complement in vitro.

In addition to the use of peptides as outlined above, the present invention relates to the peptides of the invention per se, as outlined above.

Preferred are peptides comprising or consisting of the sequence of SEQ ID NO 1 or SEQ ID NO 2, or fragments thereof, or a mutant or variant of the peptide or peptide fragment. Accordingly the invention covers fall length TORE peptides, although shorter peptide fragments are generally preferred. Preferably the peptides have complement inhibitory activity, suitably at least substantially the same as that of SEQ ID NO 1 or 2.

Most preferred are peptides containing the consensus sequence EVKI-X(n)-PY, homologous to SEQ ID NO 1, such as MSPSLVSHTQKNERGSHEVKIEHFTPY, MSPSLVSTYQKNERGSHEVKIKHFSPY, MSPSLVSDTQKHERGSHEVKIKHFSPY and HEVKIKHFSPY. Also preferred are dimeric peptides and cyclic peptides as outlined above.

Also preferred are peptides which are homologous to SEQ ID NO 2, such as SSTSDLRLMIHTKTGPIYIK (rat ed-2) and SSTSDLRLMIHTKTGPIYIK (Sm TORE ed-2), along with the C4b γ sequence TSLSDRYVSHFETEGP. Generally preferred are peptides having the general sequences [S/T]-S-x-S-D-x(0,1)-R-x-[V/M]-x-H-x-[E/K]-T-x(0,1)-G-P or X-S-X-S-D-X(0,1)-R-X(3)-H-X(2)-T-X(0,1)-G-P, where X(0,1) means either none or any.

Also particularly preferred are peptides derived from C4b capable of inhibition of complement and which are homologues to SEQ ID NO 1 or 2, for example FEVKITPGKPY.

Further preferred are peptides listed herein, capable of mediating complement inhibition.

It will be appreciated that the present invention also relates specifically to peptides which comprise the second putative extracellular domain of the TORE protein, excluding the SmTORE and ShTORE peptides. This peptide has the consensus sequence HILGFMSSTSDXRLXIHTKTGPIYIK as assessed by a comparison of the rat sequence with the two parasite sequences, with an alternative preferred consensus being SSTSDXRLXIHTKTGPIYIKST. This sequence is also available to interact with the immune system, and has a potential role in the modulation of the complement response. Further, the invention relates to mutants and variants of this sequence in which one or more amino acids are substituted, deleted or inserted.

The human TORE protein is known to co-precipitate with the fes protein, using an antibody raised to ShTORE ed-1. (Example 1.9). Fes is thought to interact with peptides containing the sequence PKYEDI, which is found at amino acid 195 in the shTore sequence, and which is conserved in the other TORE peptides. As such, the present invention also relates to TORE peptides or fragments thereof capable of interaction with human fes protein and comprising the PKYEDI consensus sequence.

Limited substitutions, deletions, insertions and inversions may be incorporated into the preferred peptides of the invention. For example, the preferred sequences: HILGFMSSTSDXRLXIHTKTGPIYIK, SSTSDXRLXIHTKTGPIYIKST, MSPSLVS, ERGSHEVKI, MSPSLVSHTQKNERGSHEVKIEHFTPY, MSPSLVSYTQKNERGSHEVKIKHFSPY, MSPSLVSDTQKHERGSHEVKIKHFSPY or MSPSLVSXTQKXERGSHEVKIXHFXPY, or other sequences as listed above, may be varied, provided that the peptide comprising the sequence retains the ability to interact with the C2 component of complement. Thus, the present invention also relates to functional equivalents of those peptides described above.

In a yet further aspect, the invention relates to pharmaceutical compositions comprising a peptide according to the present invention in combination with a pharmaceutically acceptable carrier. Preferred peptides of the invention may be delivered without any carrier, but more preferably with a suitable pharmaceutical carrier. Examples of suitable carriers are well known in the art. Preferred are compositions having the amino acid sequence EVKI-X(n)-PY, wherein “X” is any amino acid, and “n” is 1-6. Where appropriate, more than 1 peptide may be delivered for effective complement inhibition. In a further preferred aspect, the peptide or peptides of the invention may be delivered in combination with other drugs or therapies, for example, with known complement inhibitors. Suitable complement inhibitors include soluble complement inhibitors such as recombinant soluble complement receptor 1 (rsCR1) and C1-inhibitor. Other suitable complement mediators are well known in the art.

It will be appreciated that an extracellular peptide of the TORE protein may adopt different confirmation if it is not bound to a cell membrane. Therefore, we prefer that therapeutically effective peptides are delivered in the form of liposomes, for example, in which the peptide may be presented in combination with a lipid bilayer, such that the peptide is in the correct conformation.

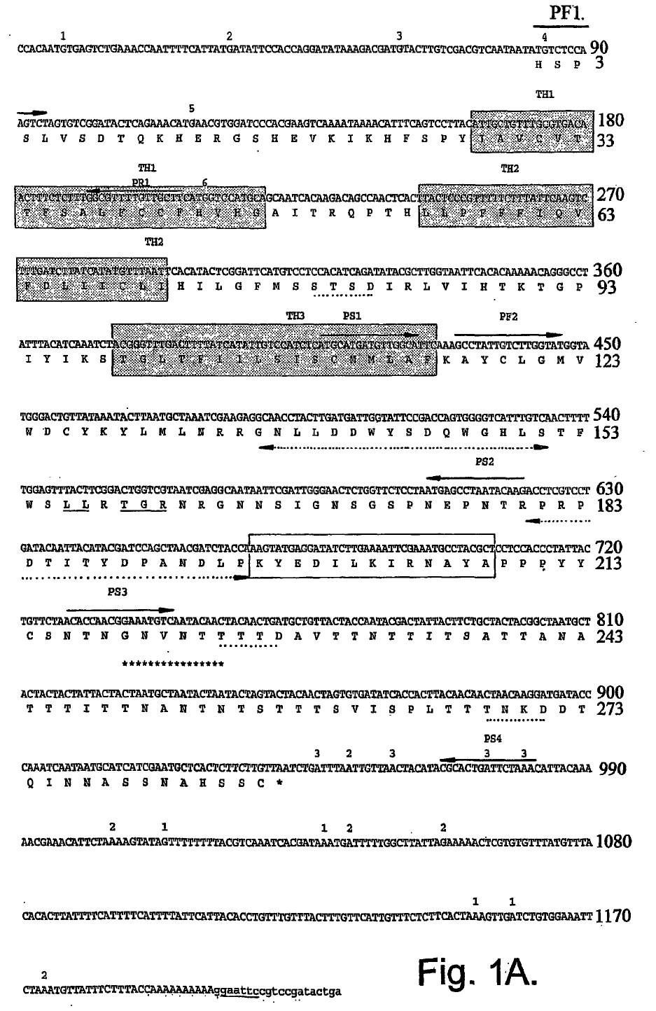

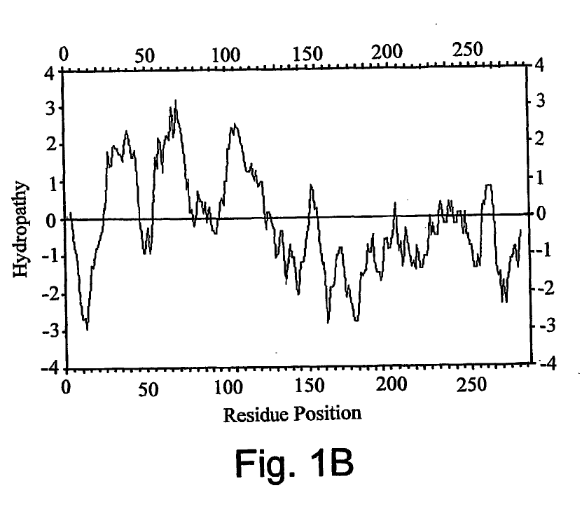

The present invention also relates to polynucleotide sequences such as DNA and RNA encoding the preferred protein and peptide fragments discussed above. The specific DNA sequences relating to the Schistosome and rat TORE proteins are given in FIGS. 1A, 18 and 20. It will be appreciated that the present invention also includes variants of the DNA sequence which encode the peptides of the invention, such as addition, deletion, inversion or substitution mutants.

In addition, the present invention relates to DNA and RNA which can hybridise with the DNA sequences of the present invention, under standard hybridisation conditions of 45° C. and 5×SSC, for example, or 60° C. and 5×SSC. Accordingly the present invention also relates to RNA for use in antisense therapies. Antisense RNA may be used to inhibit the expression of the TORE protein, such as the human TORE protein, which may increase the susceptibility of cells to complement action, for example.

The present invention also relates to polynucleotides such as DNA and RNA species which are homologous to the DNA of the present invention. We prefer that the DNA is at least 60 percent identical at the nucleotide level, more preferably 70 percent, with 80 percent identity at the nucleotide level particularly preferred.

The present invention further relates to nucleic acid sequences which are fragments of the full length DNA sequence encoding the TORE protein, especially DNA or RNA encoding the ed-1 or ed-2 domain. In addition, the invention also relates to DNA sequences which are homologous to the DNA encoding ed-1 or ed-2. We prefer that the DNA has at least 60 percent identity with the DNA encoding ed-1 or ed-2, preferably 70 percent identity and more preferably 80 percent identity.

The present invention extends to any polynucleotide sequence, such as a DNA sequence discussed above contained within a plasmid vector, optionally in combination with suitable regulatory sequences. The invention further relates to any suitable cells, either prokaryotic or eukaryotic cells, in which the vector may be maintained.

DNA or other polynucleotide encoding a member of the TORE family may be identified in a number of standard ways. Specifically, the sequence of a parasite or rat gene may be used to design primers in order to amplify homologous DNA from other species by use of the PCR reaction, for example. In addition, DNA sequences of the present invention may be used as probes in a Southern blot experiment, to identify whether a homologue exists in particular species. A suitable method is given in Example R1.6, in which fall length ShTORE cDNA was used to identify a homologue in Trypanosoma cruzi. Once a homologue has been identified by Southern blotting, a suitable DNA fragment may be cloned, preferably from an appropriate size-selected DNA library. Other suitable methods for obtaining a DNA sequence given a suitable homologous probe are well known to the person skilled in the art. Therefore, the present invention extends to a method for the identification of TORE family members, using any of the methods discussed above, which are standard in the art.

Analysis of human genomic DNA using the SmTORE cDNA under low stringency indicates the presence of human DNA homologous to SmTORE. The presence of a human homologue to the ShTORE protein has been confirmed by Western blotting using an anti-ShTORE ed-1 antibody to probe total human cell extracts. As described above, the human gene may be cloned by use of primers designed to a known TORE DNA sequence in a PCR reaction. Alternatively, an antibody to the extracellular domain may be used to screen an expression library.

The present invention also relates to antibodies raised against proteins of the present invention, particularly those antibodies raised against the N-terminal sequence (the ed-1 domain) of the TORE protein or the ed-2 domain. Suitable means for the generation of polyclonal antibodies against the ed-1 domain are given in Example R1.8.6 below. Means for generating monoclonal antibodies are standard in the art.

Antibodies, preferably monoclonal antibodies, against ed-1 or ed-2 may be used therapeutically, to block the interaction of TORE proteins with C2, thus activating complement. Antibodies may also be used therapeutically to help combat parasite invasion, by removal of cells carrying the parasite TORE protein.

A polyclonal antibody raised against the N-terminal 27 amino acids of the ShTORE protein may be used to identify equivalent proteins in other species, such as by immunoprecipitation or protein purification coupled with Western Blot analysis. Western Blot analysis using such an antibody also allows the multimeric state of the protein to be determined.

The present invention also relates to any protein that cross reacts with an antibody raised to an extracellular domain of the ShTORE protein, such as ed-1 and ed-2, preferably to a protein which is a membrane bound protein.

The present invention additionally relates to therapeutically effective preparations containing protein, peptides, DNA, RNA or antibodies as described above. Active components may be used at concentrations of, for example, 10 ng/ml to 1 mg/ml. It will also be appreciated that the therapy may be for the purpose of protection against the Schistosome or Trypanosome parasite, or may be designed to reduce inflammation, for example, by modulation of C2/C4b action.

Delivery of the peptides of the present invention may be by an suitable means such as intravenous injection. However, preferred is oral delivery of peptides, particularly short peptides of less than 15 amino acids.

The present invention additionally relates to a method of treatment of the human or animal body using peptides of the present invention, for the inhibition of human complement protein C2 activity, or modulation of human complement activity.

In particular, the invention relates to a method of treatment comprising administering an effective amount of a peptide according to the present invention to a patient in need of such treatment. Preferred is treatment of patients suffering from unregulated complement activation, for example those with autoimmune diseases such as rheumatoid arthritis, multiple sclerosis, systemic lupus erythematosus, myasthenia gravis, neuromuscular diseases (dermatomyositis [DM], Lambert-Eaton mysthenic syndrome [LEMS]), following cardiopulmonary bypass surgery; or other diseases in which complement is activated such as myocardial infarction, and Alzheimer's disease. Other diseases involving complement disfunction are well known in the art, and are included in the present invention.

In a further aspect, the invention relates to use of the peptides of the invention in xenotransplantation. Xenotransplantation has until recently been limited by the strong complement activation induced by xenospecific antibodies. In various animal models of severe complement-mediated inflammation, tissue injury has been reduced by depleting complement. Expression of membrane-bound regulators of complement activation (mRCA) in xenogeneic cells can protect in animal models of graft rejection. Accordingly peptides of the present invention may be used to reduce tissue injury in xenotransplantation.

In an alternative aspect, the TORE protein or peptide could be overexpressed in the donor organ used in xenotransplantation, for example by generation of transgenic animals with such TORE peptide overexpression. The present invention relates to such transgenic organisms and methods of treatment involving use of such tissues in xenotransplantation.

In addition to use in general complement modulation, we have determined that antibodies to TORE peptides, such as ed-1, and the use of peptides of the present invention to generate such antibodies in vivo, can help increase the anti-tumour response of the body.

Carcinoma cells express complement inhibitors to higher levels than normal cells. It is thought that this overexpression of complement inhibitors may be involved in the suppression of the in vivo anti-tumour immune response. The present invention demonstrates that anti-TORE antibody, which effectively inhibits a complement inhibitor, is able to increase complement mediated cell killing of carcinoma cells.

Accordingly the present invention relates to use of peptides of the present invention, pharmaceutical compositions comprising such peptides, and antibodies raised to such peptides in the preparation of a medicament for treatment of cancer. Specifically the invention relates to a method of treating a patient with cancer, comprising administering an effective amount of a peptide, pharmaceutical composition, nucleic acid or antibody according to the present invention, in order to increase complement activity against cancer cells.

The present invention also relates to the use of the DNA, RNA, peptides or proteins of the invention in the preparation of a medicament for the treatment of inflammation, modulation of complement action or treatment of schistosomiasis. The invention additionally relates to a method for the treatment of schistosomiasis or inflammation, comprising administration of a therapeutically effective amount of the DNA, RNA, protein or peptide of the invention, optionally in combination with a suitable adjuvant or carrier.

In particular, the present invention further provides for a TOR-based anti-schistosomal, anti-trypanosomal or anti-malarial vaccine. Western blotting using antibodies to ed-1 indicates the presence of a malarial TOR homologue possessing an ed1 motif. The vaccine is preferably in the form of a passive vaccine in which anti-TOR antibody is administered. Alternatively the vaccine may be a peptide vaccine. Suitably the peptide vaccine is able to block the TOR receptor on these parasites thereby eliminating the ability of the parasite receptor to inhibit host complement. Passive administration of anti-Sh-TOR-ed1 antibody has markedly reduced parasitaemia in trypanosoma-infected mice.

In a further aspect the invention provides for use of TORE peptides or anti-tore antibodies for detection of pathogens such as Schistosoma or trypanosoma.

The invention is further described in relation to the following Examples and Figures, which are not limiting to the present invention, wherein:

FIG. 1(A) shows the nucleotide and deduced amino acid sequence of ShTORE;

FIG. 1B shows a Kyte and Doolittle plot of ShTORE with predicted hydrophobic transmembrane and hydrophilic domains;

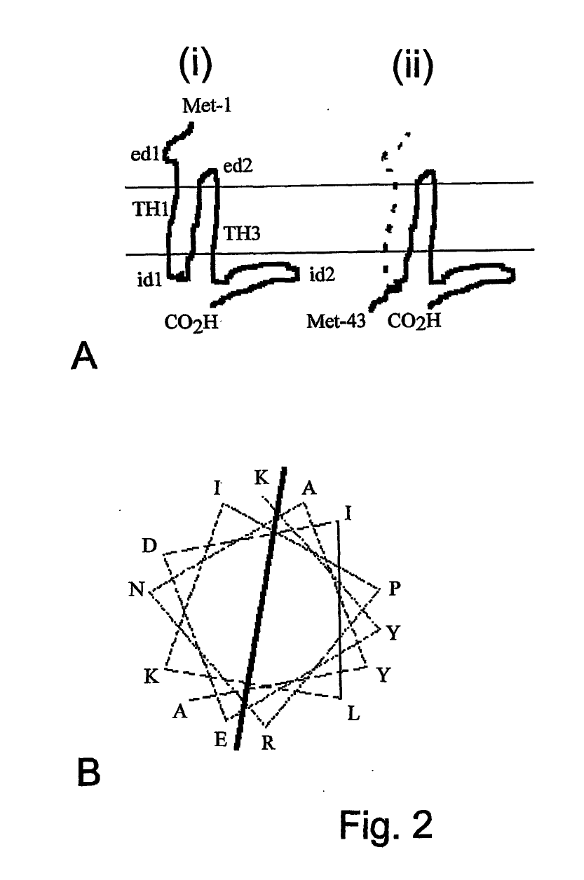

FIG. 2(A) shows the predicted membrane topology of the ShTORE molecule (i) starting at ATG4 (the 32 kDa TORE) and (ii) the predicted 27 kDa molecule from ATG6;

FIG. 2(B) is a Helical wheel representation of ShTORE-id2, Lys196-Ala208;

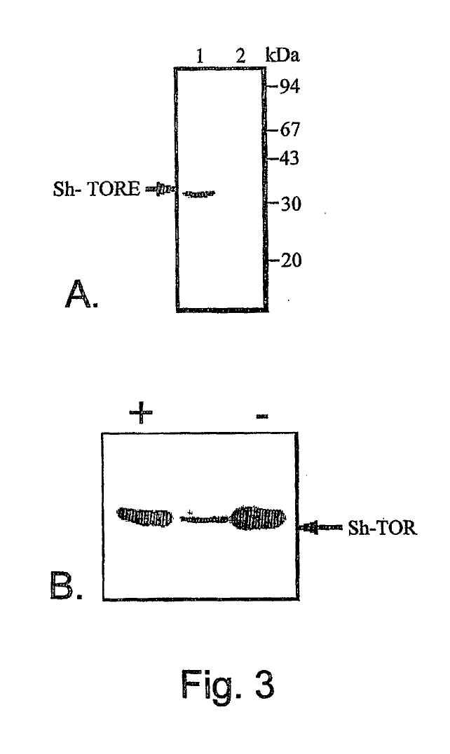

FIG. 3(A) is a Western Blot using a rabbit anti-ShTORE-ed-1 antibody as a probe against an adult worm preparation of S. haematobium;

FIG. 3(B) is a Western Blot using an anti-ShTORE-ed-1 antibody as a probe against extracts of L cells transiently expressing ShTORE, with and without exposure to tunicamycin;

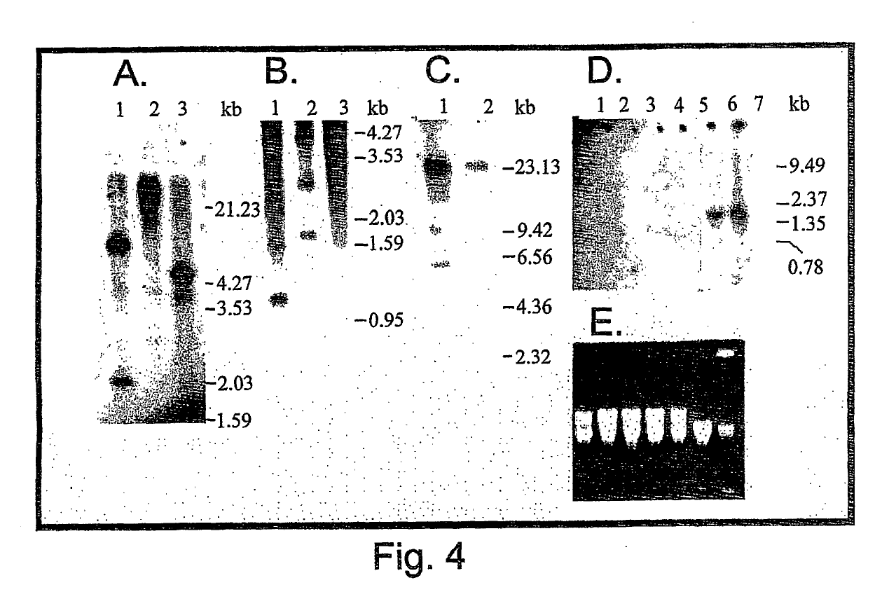

FIG. 4(A) is a high stringency Southern blot of S. haematobium genomic DNA probed with full length ShTORE cDNA;

FIG. 4(B) the blot of FIG. 4(A) probed under low stringency conditions;

FIG. 4(C) is a high stringency Southern blot of T. cruzi genomic DNA probed with full length ShTORE cDNA;

FIG. 4(D) is a Northern blot of total RNA from different stages of C. elegans and S. haematobium probed with full length ShTORE cDNA;

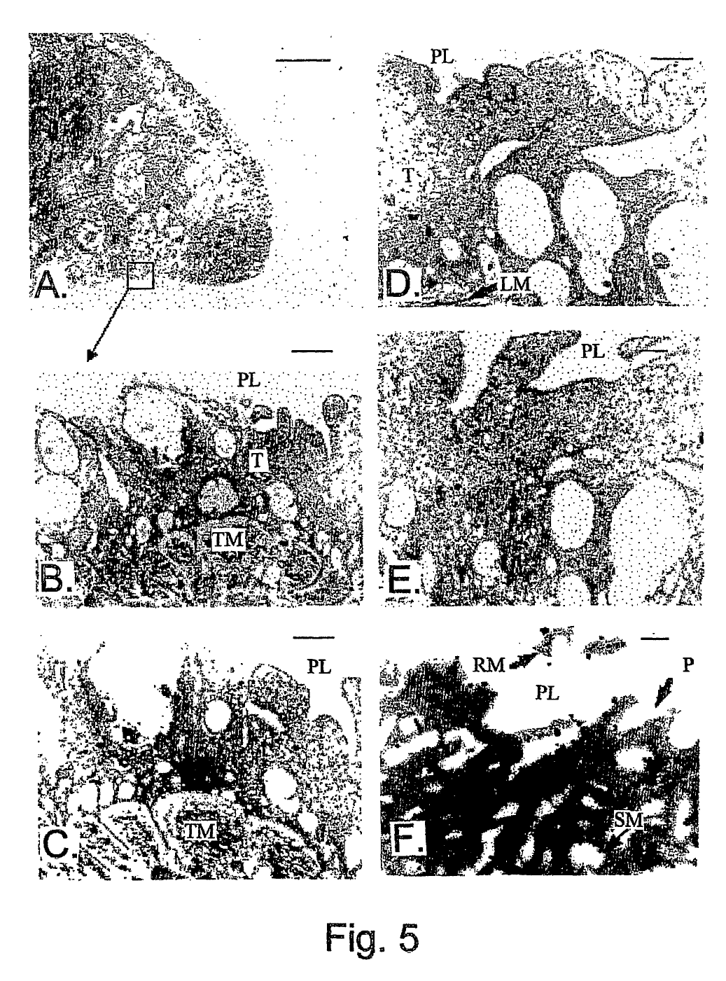

FIG. 5 demonstrates Immunoelectron microscopy of surface tegument (T) region of S. haematobium adult worms to reveal location of ShTORE;

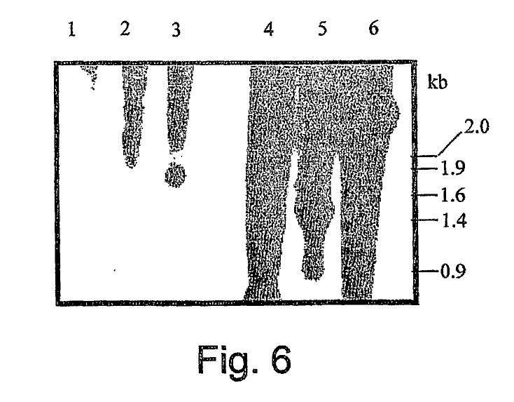

FIG. 6 is a Southern blot demonstrating the presence of a human homologue of TORE;

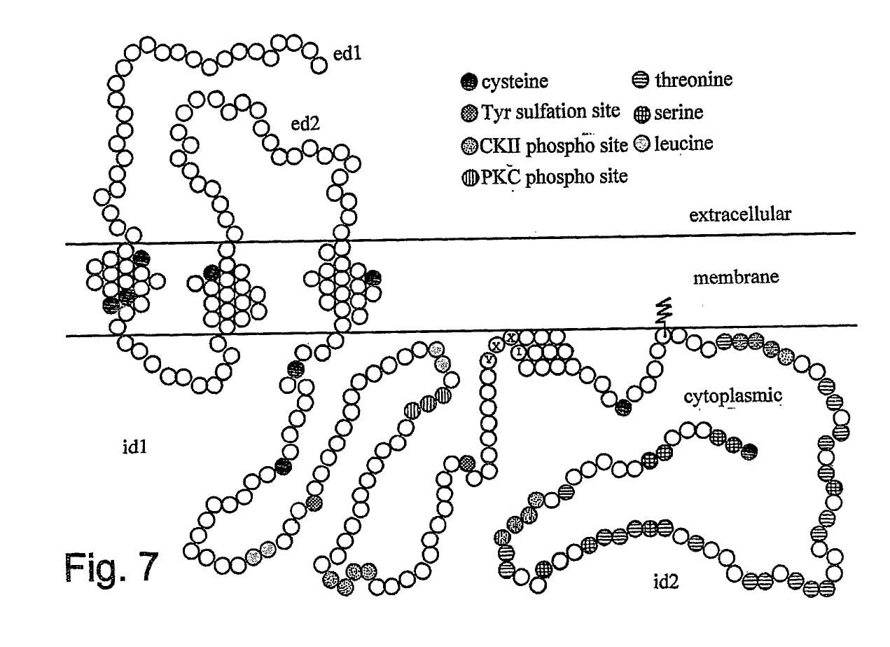

FIG. 7 is a schematic diagram to show ShTORE membrane topology;

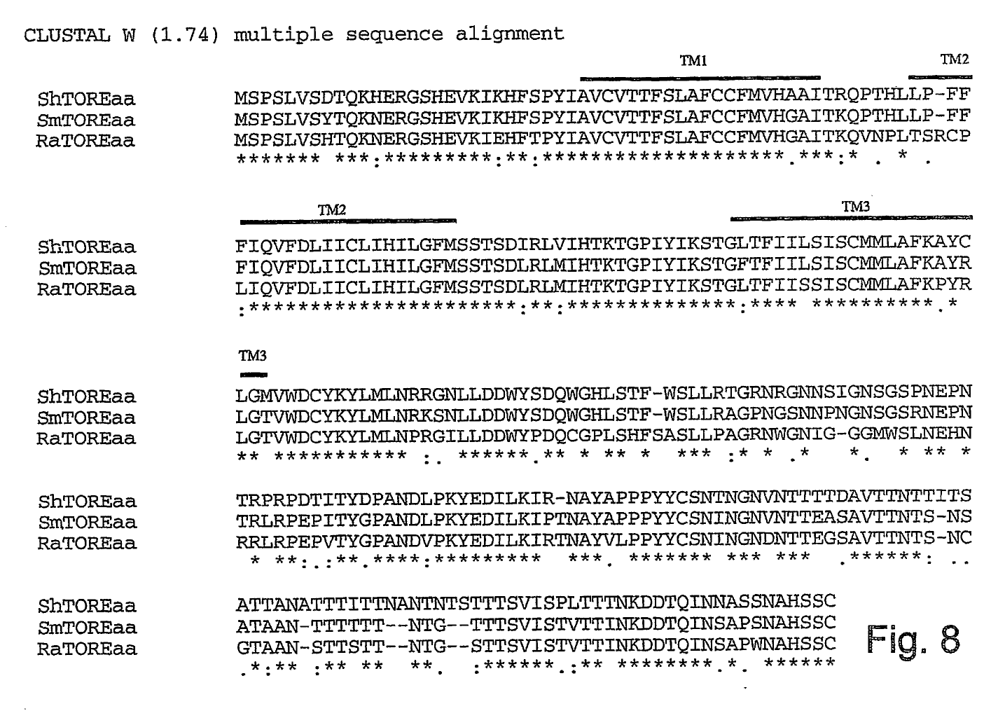

FIG. 8 shows the amino acid sequence alignment of Rat, Schistosoma mansoni and Schistosoma haematobium TORE proteins;

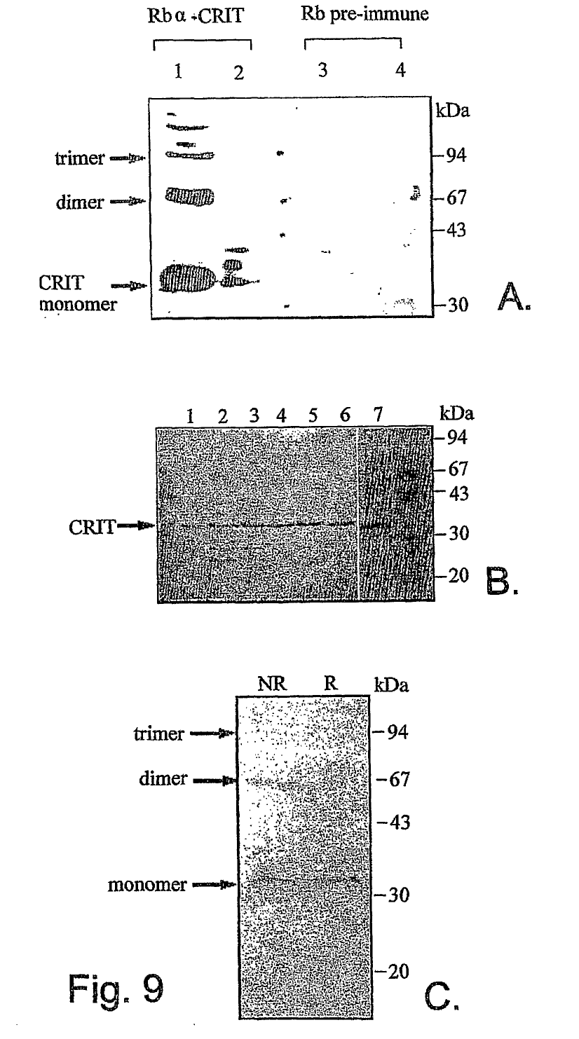

FIG. 9A shows the presence of TORE in platelets;

FIG. 9B shows the presence of a TORE homologue in a number of human cell lines;

FIG. 9C shows the multimeric structure of TORE by Western Blotting;

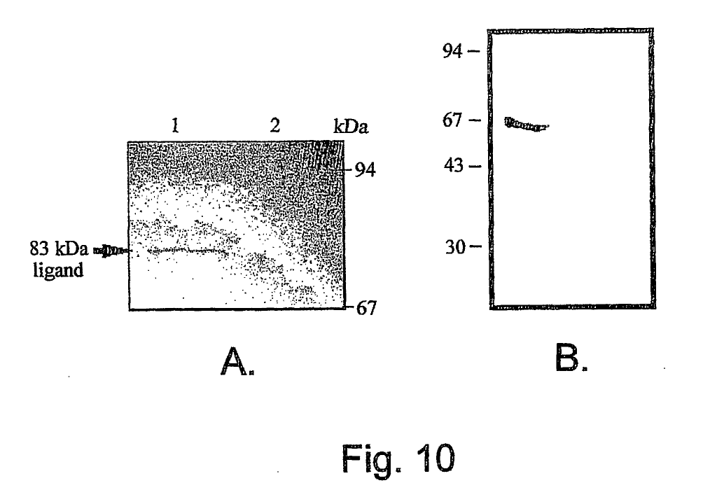

FIG. 10A shows the nature of the TORE ligand by affinity chromatography;

FIG. 10B shows binding of human complement protein C2 to the dimeric form of the putative human TORE;

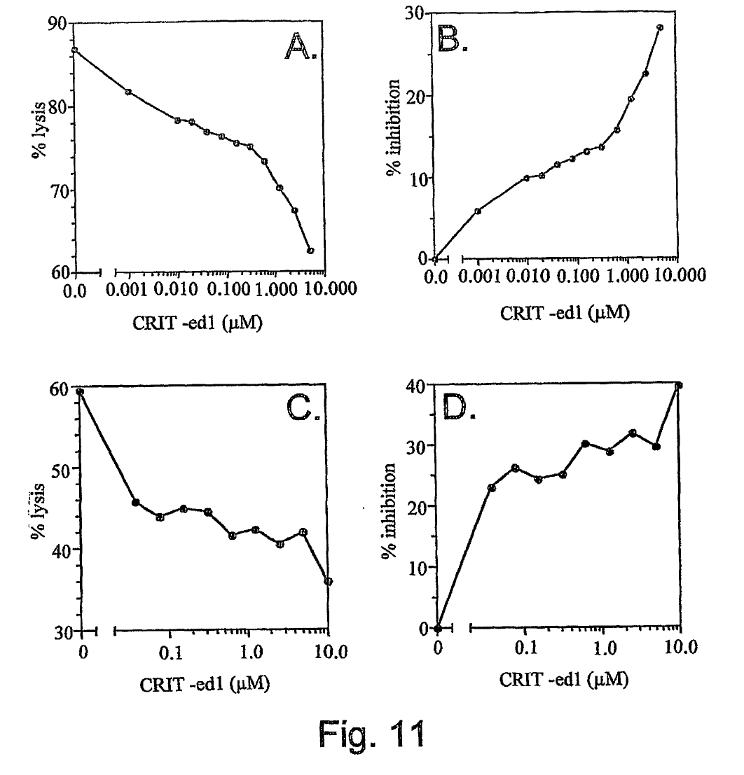

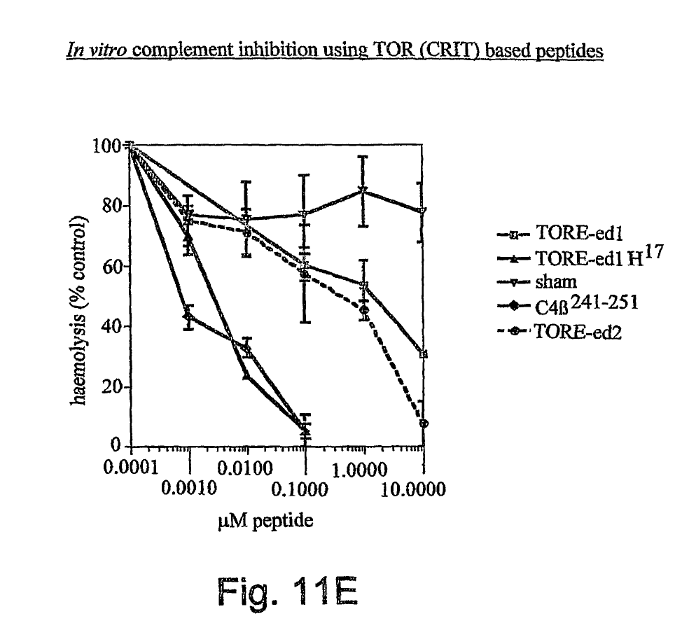

FIG. 11A-E shows the effect of the TORE based peptides on lysis of sheep red blood cells;

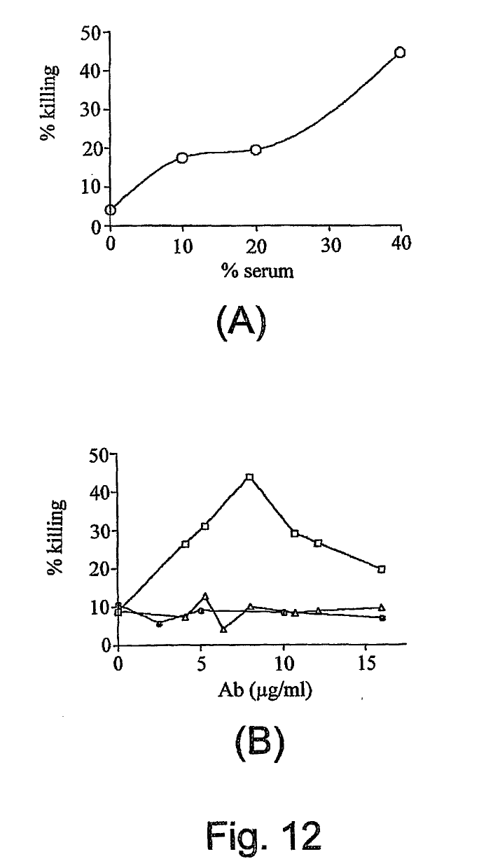

FIG. 12 shows the effect of anti-TORE-ed-1 antibody on complement mediated cell killing;

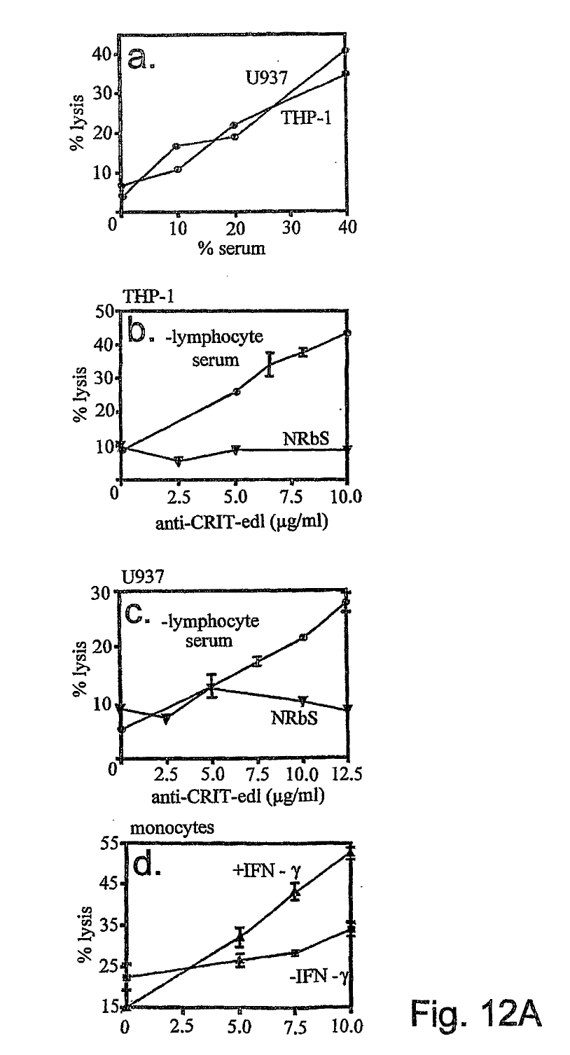

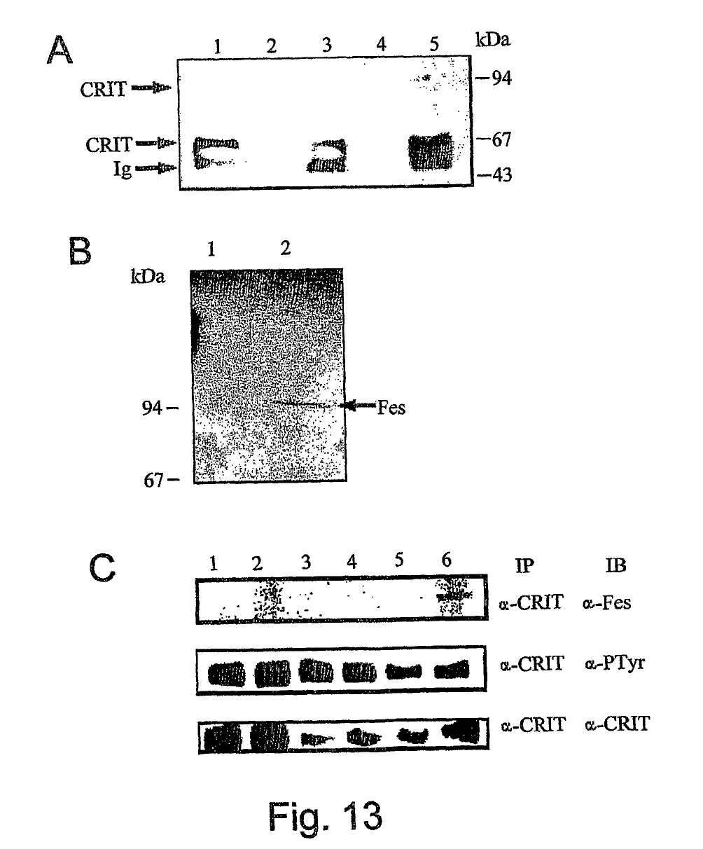

FIG. 13A indicates there is phosphorylation of TORE on THP-1 cells and Schistoma haematobium;

FIG. 13B shows co-immunoprecipitation of TORE with the fes protein;

FIG. 13C shows the effect of antibodies and endocytosis inhibitors on TORE phosphorylation and fes interaction;

FIG. 14 shows the amino acid sequence alignment of the N terminal ed-1 domains of ShTORE, SmTORE and RatTORE with two sequences from human C4b complement protein;

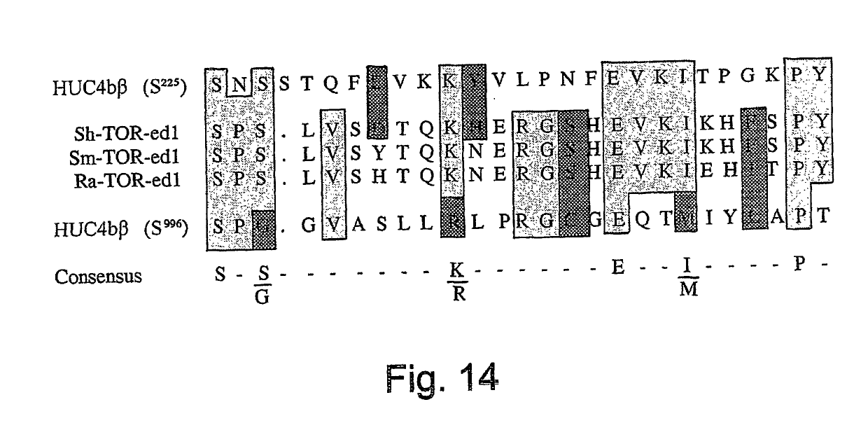

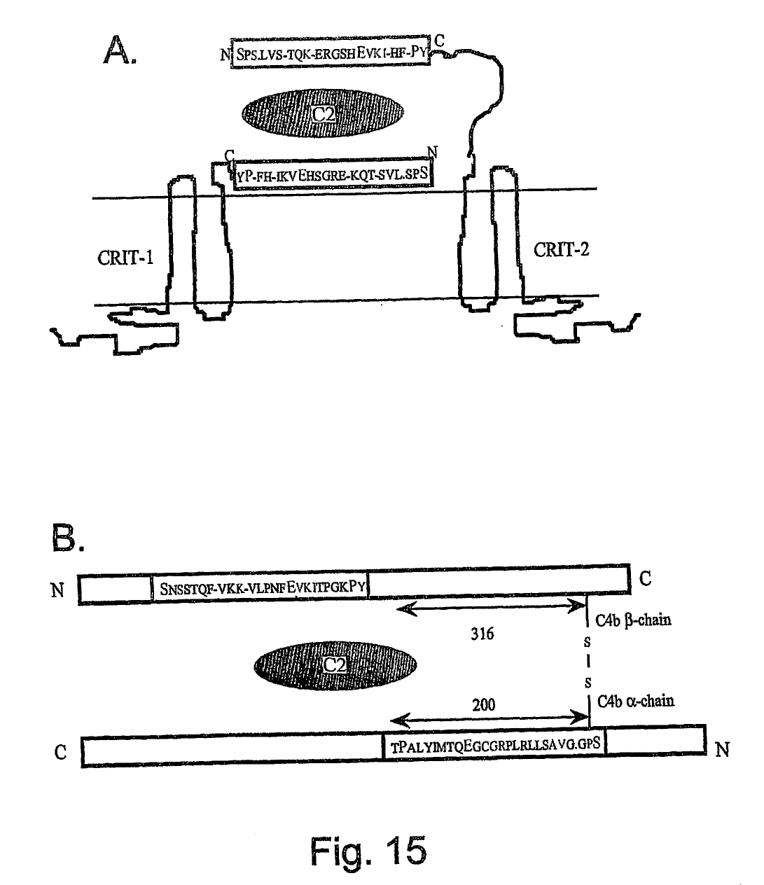

FIG. 15 is a schematic diagram showing the TORE homo-dimer bound to C2;

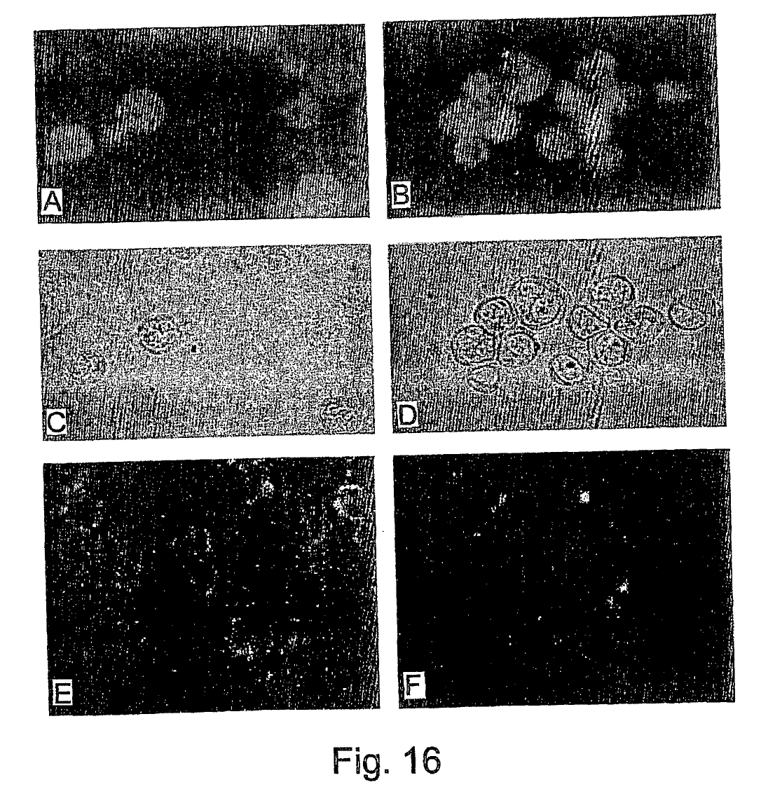

FIG. 16 shows immunofluorescence of THP-1 cells to indicate the endocytosis of TORE;

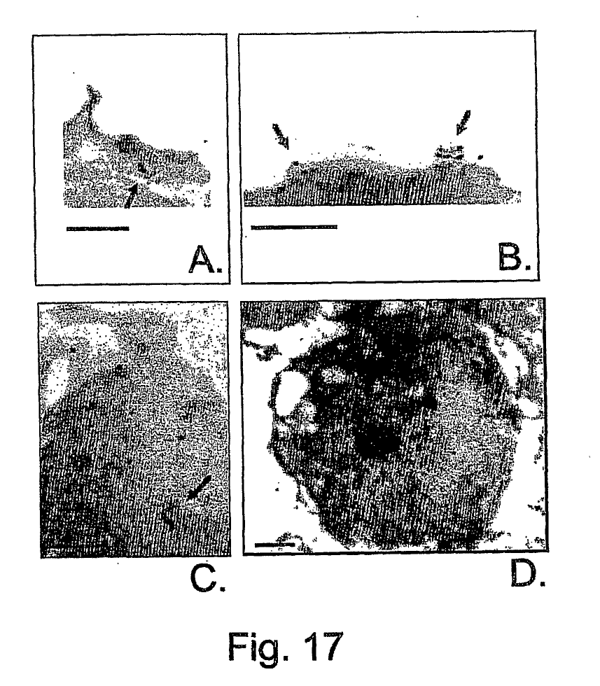

FIG. 17 shows immunoelectron microscopy of THP-1 cells to indicate TORE endocytosis;

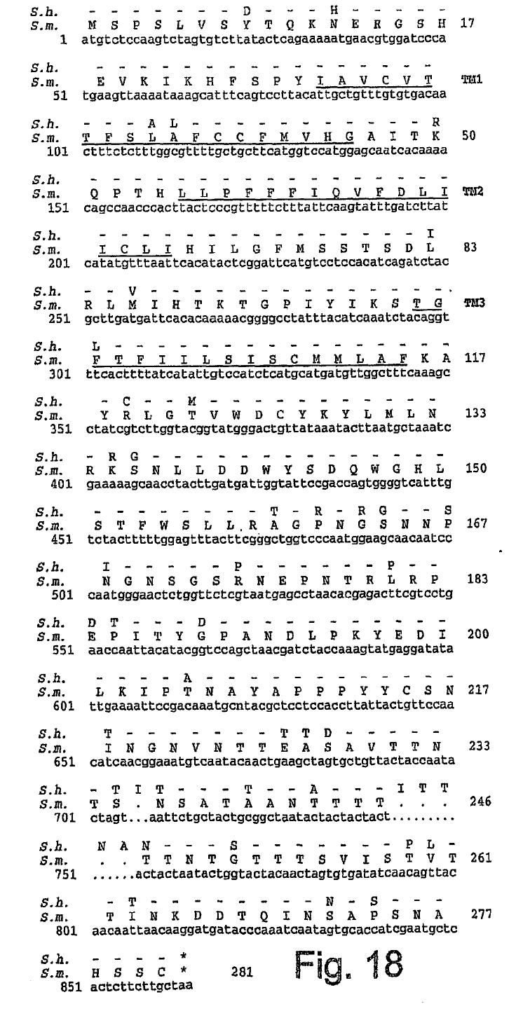

FIG. 18 shows the DNA sequence encoding the SmTORE protein;

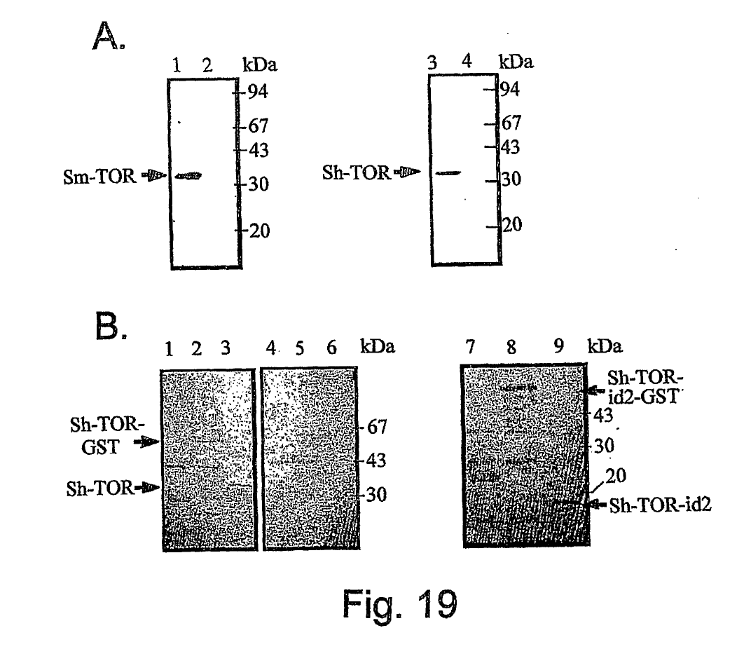

FIG. 19 shows the antibody recognition of native SmTORE and ShTORE as well as that of recombinant ShTORE by Schistosoma vaccination sera;

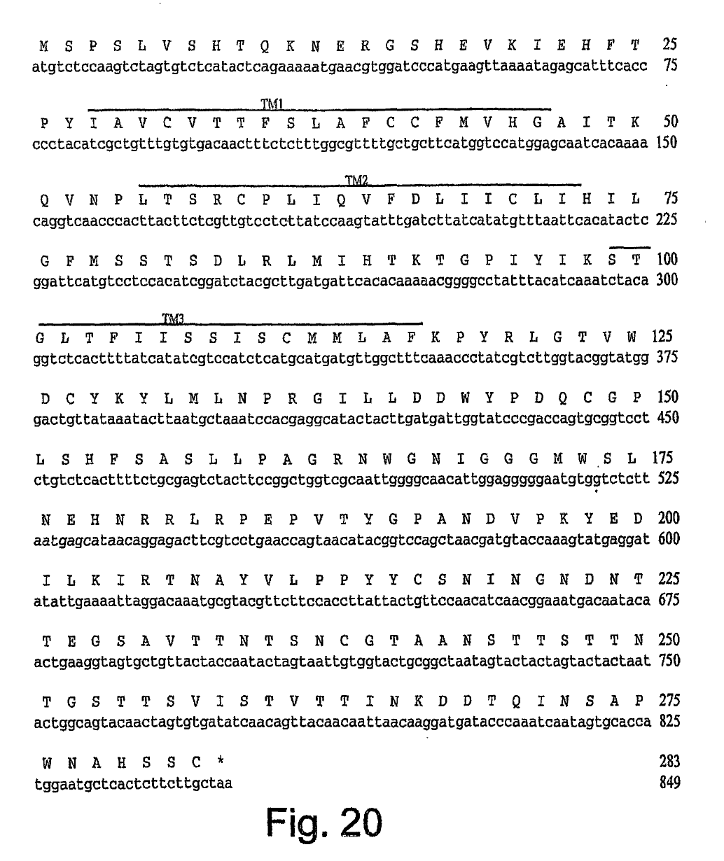

FIG. 20 shows the DNA sequence encoding the RaTORE protein;

FIG. 21 shows amino acid identity between ed-1, ed-2 and C4b; and

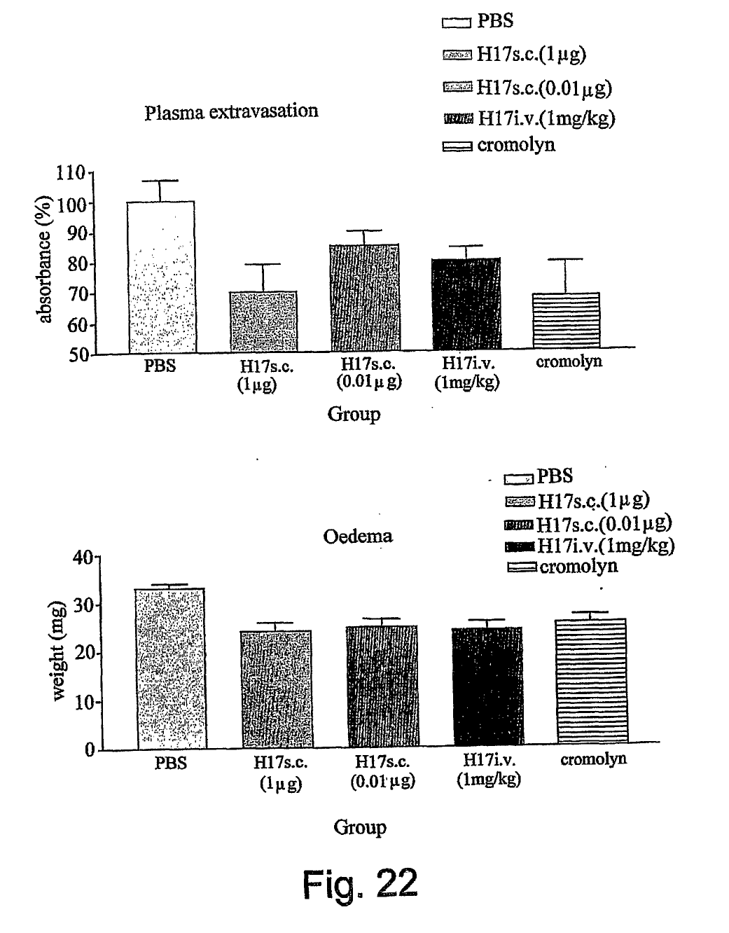

FIG. 22 shows data from in vivo studies using the peptide HEVKIKHFSPY.

### EXAMPLES

Reference Example 1 discloses the cloning and analysis of the ShTORE and SmTORE proteins. Example 1 relates to functional studies on the interaction of TORE proteins with the immune system. Reference Example 2 relates to the SmTORE protein.

### Reference Example 1

R1.1 Cloning of ShTORE from an S. haematobium cDNA Library

An adult worm S. haematobium cDNA library in λgt11 [Renganathan, E. A. et al. (1993) Trop. Med. Parasitol. 44, 187-191] was immunoscreened with VbabS. Twenty-five, triple-positive phage clones were sub-cloned into EcoRI-digested pUC18 and partially sequenced. One clone, A8, with a 1200 bp insert, showed no major homology to any other sequence in the GenBank database. As the translation of this partial sequence of the A8 clone indicated a putative transmembrane domain, at the N-terminus, it was decided to sequence fully this clone.

R1.2 Nucleotide Sequence of ShTORE

The nucleotide sequence of the full length ShTORE cDNA is presented in FIG. 1A and the hydrophobicity plot in FIG. 1B. There are 6 potential ATG start codons, but numbers 1, 2, 3 and 5 are followed by stop codons after just 1, 21, 12 and 9 codons, respectively. The two in-frame potential translational start sites, ATG start codon numbers 4 (ATG⁴) and 6 (ATG⁶), are both within a favourable Kozak sequence [Kozak, M. (1984) Nucl. Acids. Res. 12, 857-872] at nucleotide numbers 82 and 208. TAA stop codons occur 858 and 732 bp downstream of ATG⁴ and ATG⁶ respectively. From ATG⁴, the expected size of the protein would be 31.7 kDa and from ATG⁶, missing part of the N-terminus, 27.1 kDa (FIG. 2A). After the TAA stop and within the first 232 bp of the A/T-rich 3′-UTR, there are 4 more in-frame (reading frame 1) stop codons as well as 9 more in the remaining 2 reading frames.

R1.3 Deduced Amino Acid Sequence of ShTORE

The nucleotide sequence of ShTORE translates into a predicted protein of 286 amino acid residues, corresponding to a relative molecular mass of 31.7 kDa, which shows no significant homology with any other protein in the SWISSPROT database. The protein lacks a signal peptide, instead having an N-terminal hydrophilic domain, ed-1, of 27 residues, which is predicted as being extracytoplasmic [Rost, B. (1996) Meth. Enzymol. 266, 525-539 and Rost, B. et al. (1995) Prot. Sci. 4, 521-539]. ShTORE has 3 N-terminal transmembrane (TM) domains, TM1, TM2 and TM3 as indicated in the hydrophobicity plot (FIG. 1B) and in FIG. 2A. These 3 hydrophobic domains are predicted as being transmembrane on the basis of each comprising stretches of 17 hydrophobic amino acid residues (hydrophobic index>2.5) which, according to the Chou-Fasman algorithm (K) would form an alpha-helix and because they are flanked by charged residues. The short intervening hydrophilic domains, id1 and ed2, are also indicated in FIG. 2A (i), as well as the 171 residues long C-terminal, predicted intracytoplasmic [Rost, B. (1996) Meth. Enzymol. 266, 525-539 and Rost, B. et al (1995) Prot. Sci. 4, 521-539] hydrophilic region, called id2. Within id2, which comprises Lys¹¹⁶ to Cys²⁸⁶, there are predicted consensus phosphorylation sites for protein kinase C [Woodgett, J. et al (1986) Eur. J. Biochem. 161, 177-184] on Thr¹⁵⁹ and Thr²⁶⁸ and casein kinase II [Pinna, L. (1990) Biochim. Biophys. Acta 1054, 267-284] on Ser¹⁷³, Thr224 and Thr268. The id2 domain also possesses a putative myristoylation site at Gly²¹⁹ and has a total of 9 Tyr residues. ShTORE has no tyrosine kinase catalytic domain or other enzyme motif.

Non-empirical evidence based on sequence homology suggests regions within the cytoplasmic tail, id2, that could be involved in the internalization of ShTORE. The secondary structure of id2 predicts a region from Lys¹⁹⁶ to Ala²⁰⁸ with a high probability of forming an alpha-helix which terminates with consecutive prolines Pro²⁰⁹Pro²¹⁰Pro²¹¹. A helical wheel representation of Lys¹⁹⁶-Ala²⁰⁸, (FIG. 2B) shows the helix to divide symmetrically into a predominantly hydrophobic half (with some aromatic side chains) and the other mainly basic. Within this amphipathic helix there is the sequence Y¹⁹⁷EDI, analogous to the internalization motif YXXφ, where φ is an amino acid with a large hydrophobic group. A similar helical structure in the G-protein coupled receptor (GPCR), angiotensin II, is used in agonist-stimulated endocytosis [Thomas, W. G., et al (1995) J. Biol. Chem. 270, 22153-22159]. It is possible that the hydrophobic side of the amphipathic helix has either a loose association with the membrane or that it interacts with another protein(s), possibly at the candidate phosphotyrosines Tyr¹⁹⁷ and Tyr²⁰⁷. Interestingly, Tyr¹⁹⁷ lies within the putative SH2-binding site, YEDI, which could potentially associate with the protein tyrosine kinases (PTK), Shc, fes/fips or Syk [Songyang, Z., et al. (1994) Mol. Cell. Biol. 14, 2777-2785 and Songyang, Z., et al. (1995). Nature 373, 536-539]. In schistosomes, a homologue of the human Fer (PTK) gene has been previously isolated by PCR using degenerate oligonucleotides based on the conserved PTK catalytic domains VI and VIII (J.Inal, unpublished data). N-terminal to Y¹⁹⁷ is the motif LPKY¹⁹⁷. A similar β-turn motif, NPXY, in human erb-B2, mouse TrkB-1 (BDNF receptor), melanoma receptor and mouse CD3 is predicted to form a bend [Bansal, A. and Glerash, L. (1991) Cell 67, 1195-1201] which helps present the phospho-Tyr motif for binding to the SH2 domain of the PTK. The process of tyrosine phosphorylation of receptors lacking kinase activity by cytoplasmic kinases, in response to agonist, is thought to play a part in endocytosis [Thomas, W. G., et al (1995) J. Biol. Chem. 270, 22153-22159]. Within ShTORE-id2 there are also two di-Leucine motifs at Leu¹³⁸Leu¹³⁹ and Leu156Leu¹⁵⁷ which, from the studies of other receptors, may also be involved in receptor internalization [Sandoval, I. V. and Bakke, O. (1994) Trends Cell Biol. 4, 292-297].

The terminal 64 residues of ShTORE-id2 have an amino acid composition that is 52% (33 out of 64 residues) Ser/Thr. Serine and threonine residues, apart from being phosphorylated commonly have O-linked addition of the monosaccharide N-acetylglucosamine (GlcNAc). Although there is no strictly defined site of addition of GlcNAc, it appears that a proline, such as Pro264 in id2, within three residues of the target serine or threonine is required [Haltiwanger, R., et al. (1992) Biochem. Soc. Trans. 20, 264-269]. This modification is common in all eukaryotes, including schistosomes [Nyame, K., et al. (1987) J. Biol. Chem. 262, 7990-7995] and occurs mostly in nucleoplasmic but also cytoplasmic proteins [Haltiwanger, R., et al. (1992) Biochem. Soc. Trans. 20, 264-269]. In ShTORE-id2, Tyr¹⁴³ and Tyr¹⁸⁸ lie within consensus sequences for tyrosine sulfation [Huttner, W. B. (1988) Aim. Rev. Physiol. 50, 363-376]. This post-translational modification occurs in the trans Golgi but is quite rare in membrane proteins [Hille, A. and Huttner, W. B. (1990) Eur. J. Biochem. 188, 587-596].

R1.4 ShTORE is a Functional Protein: Immunoblot Recognition of Native ShTORE with anti-ShTORE-ed-1

The first 81 nucleotides of the open reading frame from the translational start point, ATG⁴ code for amino acid residues 1-27. A peptide of this sequence was used to raise a rabbit polyclonal antibody which gave titres of up to 1:10,000 in an enzyme-linked immunosorbent assay [Harlow, E. and Lane, D. P. (1988) Antibodies: A Laboratory Manual, Cold Spring Harbor Press, Cold Spring Harbor, N.Y.] against immobilized peptide. This antibody, anti-ShTORE-ed-1, was used to probe by Western blotting a total protein extract of S. haematobium adult worms run under reducing conditions. The expected size of the native molecule, based on the open reading frame length was 31.7 kDa, and the actual protein band recognised was of approximately 32 kDa (FIG. 3A, lane 1). The same extract probed with the rabbit pre-bleed is shown in lane 2.

The open reading frame of ShTORE (codons 1-286, also including 261 bp of 3′-UTR) was also expressed as a recombinant GST fusion protein in bacteria and shown to be specifically recognised by sera from schistosome-immunized animals (FIG. 19B).

R1.5 Transfection of Mammalian Cells with ShTORE Reveals Membrane Topology

In order to confirm whether the N-terminal hydrophilic domain of ShTORE, termed extracellular domain 1, ed-1, is really extracellular, as predicted from the amino acid sequence, the full length ShTORE cDNA was transfected into mouse fibroblast L cells. The transfected cells were then viewed by fluorescence microscopy after incubation with anti-ShTORE-ed-1, the rabbit antibody raised against the predicted extracellular domain. As only the transfected cells showed surface fluorescence with this antibody (not shown) this further supports the membrane topology for ShTORE depicted in FIG. 2A. Furthermore, if id2 really is intracellular, then none of the 6 N-linked glycosylation sites predicted in id2 (not indicated in FIG. 1A) would be utilized. If id2 is extracellular it seems likely that at least one of the six sites would be utilized. Therefore the glycosylation status of ShTORE could also give an indication of its likely membrane topology. The L cells transfected with ShTORE were therefore treated with tunicamycin. This resulted in no size difference as seen by immunoblot analysis of the 32 kDa ShTORE band, compared with the untreated transfectants (FIG. 3B). Interestingly, the truncated version of ShTORE, missing ed-1 and TM1, if a genuinely expressed product, would be predicted to have an identical topology [FIG. 2A, (ii)].

R1.6 Genomic Organization of ShTORE and Species Distribution

From the size of the mRNA transcript, 1.35 kb (see below), compared to the ShTORE cDNA sequence available, 1.2 kb, it is possible that the 10 adenine residues at the 3′ end of the ShTORE cDNA sequence represent a poly(A) tail, although there is no consensus polyadenylation sequence immediately upstream. To look at the ShTORE genomic organization, a Southern blot was carried out in which schistosome genomic DNA digested with EcoRI, HindIII and PstI was probed with full length ShTORE cDNA (FIG. 4A and B). The probe did not possess sites for the enzymes used and hybridization under high stringency conditions (FIG. 4A) resulted in one strongly hybridizing band and one additional weakly hybridizing band in the HindIII and EcoRI digests. When the Southern blot hybridization was carried out at low stringency, additional weak bands were seen (FIG. 4B), suggesting the existence of possible ShTORE subtype(s) or other receptor(s) related to ShTORE. Southern blot analysis of Trypanosoma cruzi genomic DNA under high stringency conditions with a full length ShTORE cDNA probe revealed several HindIII fragments (FIG. 4C, lane 1). A T. cruzi cosmid library in the vector pcosTL [Kelly, J. M., et al. (1994) Mol. Biochem. Parasitol. 65, 51-62] was therefore screened at high stringency with the ShTORE cDNA probe and four positive clones sub-cloned into pGem-1λT.

Northern blot analysis of C. elegans total RNA showed (FIG. 4D, lanes 1-5) that there was no TORE homologue expressed in any of the four larval stages, C1, C2, C3 or C4, nor in the adult worm. Up until the present, no homologue has been found by searching the C. elegans genome database (ACeDB), nor in any of the complete or partially sequenced prokaryotic databases.

R1.7 Stage Specificity of ShTORE and Subcellular Localization

Northern blot analysis (FIG. 4D) showed ShTORE to be expressed as a 1.35 kb transcript, more abundant in the larval stage (lane 7) than in the adult worm stage (lane 6). With the anti-ShTORE-ed-1 antibody, immunoelectron microscopy was used to localize ShTORE in adult worms. ShTORE was thereby found exclusively in the surface tegument area (FIG. 5C, E and F) the cytoplasmic area which completely surrounds the worm, with a preponderance of labelling in the tegumental plasma membrane on the surface and in the surface pits and the interconnecting channels into which they lead (FIG. 5C, E and F). Within these surface pits it has been shown in vivo [Bruce, J. I., et al (1971) Exp. Parasitol. 30, 165-173] that they all contain host blood plasma. FIG. 5A and B shows no labelling of a similar section incubated with the same antibody pre-absorbed with the ShTORE-ed-1 peptide. The section in FIG. 5D was incubated with pre-immune serum.

R1.8 Materials and Methods

R1.8.1 Nucleic Acid Purification

Genomic DNA isolation [McCutchan, T. J., et al (1984) Proc. Natl. Acad. Sci. USA. 81, 889-893] from S. haematobium worms and total RNA isolation [Sambrook, J., et al (1989) Molecular cloning: a laboratory manual, 2nd edn, Cold Spring Harbor Laboratory, Cold Spring Harbor N.Y.] from S. haematobium larvae and adult worms were performed using standard procedures.

R1.8.2 DNA Sequencing, Southern and Northern Blotting

The λgt11 clone containing the 1.2 kb ShTORE cDNA was identified by library screening using VBabS (Vaccinated Baboon Serum) obtained from baboons vaccinated with γ-irradiated S. haematobium cercariae. The ShTORE cDNA was subcloned into pUC18, to generate pUC-ShTORE. Sequencing of this construct was carried out by the dideoxynucleotide chain termination method using ³⁵S-dATP and sequenase (United States Biochemicals, Ohio, U.S.A.). M13 forward and reverse primers were used, as well as ShTORE-specific primers. All sequences were determined on both strands. Southern and Northern blotting was carried out under both high and low stringency conditions using standard protocols [Sambrook, J., et al. (1989) Molecular cloning: a laboratory manual, 2nd edn, Cold Spring Harbor Laboratory, Cold Spring Harbor N.Y.].

R1.8.3 Transfection of Mammalian Tissue Culture Cells with ShTORE

ShTORE cDNA was cleaved from pUC-ShTORE with EcoRI and subcloned into EcoRI-cut and dephosphorylated pcDNA3 (Invitrogen) to generate pc-ShTORE. The correct orientation and reading frame were confirmed by PCR and DNA sequencing, respectively. For transient transfection, the cells were 85% confluent. 20 μg of pc-ShTORE and pcDNA3 control, both purified using the Qiagen midi plasmid kit, were transfected into L cells using lipofectamin (Sigma) according to the manufacturer's instructions. To determine whether ShTORE has N-linked glycosylation, transfected cells were incubated with 10 μg/ml tunicamycin for 24 h before harvesting for Western blot analysis.

R1.8.4 Antigens

A deoxycholate extract of S. haematobium adult worm antigen was prepared by sonicating adult worms in PBS. After centrifuging at 16,000 g for 30 min, the pellet was resuspended in 2 volumes PBS by sonication and an equal volume of 4% sodium deoxycholate in PBS, added. After 1 h on ice the extract was centrifuged as above and the supernatant recovered. A recombinant antigen in the form of a GST-ShTORE fusion protein was prepared as outlined in section 3.4.

R1.8.5 Electrophoresis, Immunoblot Analysis and Library Immunoscreening

SDS-PAGE was carried out on 10% or 15% polyacrylamide gels [Laemmli, U. K. (1970) Nature 227, 680-685]. For immunoblotting, proteins were then electrophoretically transferred onto nitrocellulose membranes (Schleicher and Schuell) following standard procedures [Towbin, et al. (1972) Proc. Natl. Acad. Sci. USA 76, 4350-4354]. Blots were first probed with an appropriate dilution of the primary antibody and bound antibody then detected with horseradish-peroxidase conjugated anti-human, anti-rabbit or anti-mouse IgG antibodies (Bio-Rad) at a 1/3000 dilution and developed using diaminobenzidine with CoCl₂ or else by chemiluminescence (ECL, Amersham). Library immunoscreening was according to standard procedures [Sambrook, et al. (1989) Molecular cloning: a laboratory manual, 2nd edn, Cold Spring Harbor Laboratory, Cold Spring Harbor N.Y.] and probing of plaque lifts was essentially as above for Western blots, using VBabS.

R1.8.6 Affinity Purification of Monospecific Anti-ShTORE-ed-1 Antibodies

The ShTORE-ed-1 27-mer peptide (NH₂-Met-Ser-Pro-Ser-Leu-Val-Ser-Asp-Thr-Gln-Lys-His-Glu-Arg-Gly-Ser-His-Glu-Val-Lys-Ile-Lys-His-Phe-Ser-Pro-Tyr-CO₂H) was synthesized on an Applied Biosystems peptide synthesizer. It was purified by HPLC on a preparative C₁₈ reverse-phase column and then analysed using an amino acid analyser. A polyclonal antibody was obtained by immunizing a New Zealand white rabbit with 3×1 mg doses of the ShTORE-ed-1 peptide on days 1, 14 and 28. Affinity purification of the polyclonal antibody was then carried out according to standard protocols using an epoxy-activated Sepharose column (Pharmacia) coupled with the ShTORE-ed-1 peptide at a concentration of 2.5 mg/ml.

R1.8.7 Homology Search

The ShTORE sequence analyses and comparison with all the entries in the SWISS-PROT data base was carried out using the programme for the University of Wisconsin Genetics Computer Group (GCG) version 7.0 sequence package [Devereux, J., et al. (1984) Nucleic Acids Res.12, 387-395].

R1.8.8 Immunofluorescence and Immunoelectron Microscopy

The transiently transfected murine L cells were fixed 48 h after transfection with 3% paraformaldehyde in PBS for 15 min. The cells were then blocked in a 1% solution of BSA in PBS and treated with primary antibody in blocking buffer for 1 h. After washing for 15 min with PBS, the cells were incubated in fluorescein isothiocyanate-labelled goat anti-rabbit (Sigma) antibody for 45 min. Finally the cells were extensively washed and mounted in a 90% solution of glycerol in 50 mM Tris, pH 8, containing 1 mg/ml p-phenylenediamine and viewed by inverted fluorescence microscopy (Zeiss).

For electron microscopy, the S. haematobium adult worms were fixed in 2% paraformaldehyde and 0.1% glutaraldehyde. After extensive washing and embedding in LR Gold for UV polymerization, longitudinal sections, 90 nm thick were cut on a Reichert Jung Ultracut microtome. On Pioloform coated nickel grids, the sections were blocked, probed with rabbit anti-ShTORE-ed-1 at a 1/600 dilution (1.3 μg/ml), and a gold-labelled goat anti-rabbit IgG (Sigma). After silver enhancement [Danscher, G. (1981) Histochemistry 71, 1-16] and staining in uranyl acetate, the sections were dried and then observed using a JEOL 1299EX transmission electron microscope.

### Example 1

1.1 A Putative Human Homologue of TORE

Southern blotting, under low stringency conditions (45° C. for 16 h in 5×SSC, using the full length SmTORE cDNA as a probe, 2 washes with 3×SSC, 0.1% SDS at room temperature, rinsing in 2×SSC and exposure to X-ray film with an intensifying screen for 48 h) revealed the likelihood of a human TORE homologue, HuTORE (FIG. 6). It was also found (section 2.3) that the antibody raised against the ShTORE N-terminal extracellular domain, ed-1, recognised an identically sized 31-32 kDa protein. This is likely to be HuTORE because it also binds to the same ligand as does ShTORE (section 2.6).

1.2 Alignment of Schistosoma Species and Rat Amino Acid Sequences of TORE

The Rat TORE was cloned by PCR using degenerate primers based on the sequence of the Schistosoma TORE (STORE). The primers used were as follows:

| | | |
|---|---|---|
| Sense: | 5′-CGCGATGTC(C/T)CC(A/C/G/T)I(C/G)ICTIGTITC-3′ | (16-fold degenerate) |
| Antisense: | 5′-CGCGTTA(G/A)CAIGAIGA(G/A)TG(A/C/T/G)GC(A/G)TT-3′ | (16-fold degenerate). |

The PCR cycling was carried out at 94° C. (30 seconds), 55° C (60 seconds) and 72° C. (60 seconds), for 35 cycles.

The DNA fragment isolated was cloned into pGem-T and sequenced in both strands.

FIG. 7 shows how ShTORE, representative of the other sequences, would be expected to lie in the membrane and shows some of the conserved protein motifs. An alignment of the two Schistosoma and the rat TORE sequence is presented in FIG. 8. SmTORE and ShTORE have 87% identity at the amino acid level and 91% similarity. SmTORE and RaTORE have 80% identity and 84% similarity. ShTORE and RaTORE have 75% identity and 80% similarity. The majority of the changes between RaTORE and STORE occur immediately after the third transmembrane domain, within the first 60 residues of the 172 residue long cytoplasmic tail, although most of the endocytosis motifs, consensus phosphorylation sites and other features are shared by all three sequences.

Comparison of the putative ligand-binding, extracellular domain 1, (ed-1), of the sequences obtained, as shown in FIG. 8 indicates the high degree of homology with ed-1. Out of the 27 residues making up ed-1, there is 92% amino acid identity between ShTORE ed-1 and SmTORE ed-1, 89% identity between SmTORE ed-1 and RaTORE ed-1 and 85% identity between ShTORE ed-1 and RaTORE ed-1. The high identity between species suggests that the human TORE protein has a very similar sequence to the rat and Schistosome proteins. This explains the cross-reacting anti-ShTORE ed-1 antibody which, as well as the STORE, also recognises the HuTORE protein in Western blotting, FACS analysis and immunofluorescence and immuno-electron microscopy. As a result of the biological cross reactivity it is very likely that the HuTORE ed-1 sequence and ShTORE ed-1 sequence are at least 75% identical, else such cross reactivity would not be observed.

1.3 Cellular Distribution of TORE, a Receptor which Forms Oligomers

Western blotting FIG. 9B and FACS analysis showed expression of TORE in various human haematopoietic cell lines. Identification of the identically sized ShTORE is shown in lane 7 for comparison. The protein extracts in FIG. 9B are all run under reducing conditions. FIG. 9C shows that TORE is able to form covalent oligomers. These disulphide-linked dimers and trimers remain bound when separated by SDS-PAGE, run under non-reducing conditions (FIG. 9C, lane 1). When the same extract was run under reducing conditions, there was an increase in intensity of the monomeric form, and a disappearance of the dimeric and trimeric forms (lane 2).

As well as in various carcinoma cell lines, TORE was shown to be expressed in various normal cells, albeit at a lower level of expression, including human platelets (FIG. 9A, lane 2) and normal lymphocytes (not shown).

1.4 Identification of a Ligand for ShTORE by Receptor Affinity Chromatography

In order to identify a likely ligand, the putative ligand-binding domain of ShTORE, extracellular domain 1 (ed-1), was used in receptor affinity chromatography. It was assumed that the cells expressing the receptor themselves produced the ligand in an “autocrine” manner. The cells chosen were the macrophage-like (human) THP-1 cells. These were adapted to grow in conditioned, serum-free medium in order to increase the chances of finding the ligand for TORE. One litre of serum-free medium which had maintained the cells, and as a control one litre of serum-free medium without cells, was passed through identical epoxy-activated Sepharose 6B columns with the 27-mer ShTORE-ed-1 peptide attached. Fractions were collected from a low pH elution and 20 μl aliquots run on SDS-PAGE. An 83 kDa protein band was identified (FIG. 10A, lane 1). The fraction was concentrated, transferred to problot and stained with Coomassie Brilliant blue. The band was then excised and the N-terminal sequence determined. The resulting six residues indicated the protein to be human complement protein C2. The fully glycosylated version of C2, however should have been 102 kDa. The non-glycosylated form of C2 was in fact identified, because the cells were grown in serum-free medium and, under such conditions, cells do not produce fully mature, glycosylated proteins.

1.5 Western Blotting with Anti-Complement Protein Antibodies

C2 of the Classical complement pathway and Factor B of the Alternative complement pathway show 39% identity at the amino acid level and across the six residues obtained by N-terminal sequencing of the putative ligand would show just one amino acid difference. Western blotting of the fraction containing the 83 kDa protein, concentrated 100-fold using a Centricon spin column, was therefore carried out using anti-C2 and anti-Factor B antibodies. Recognition of the 83 kDa protein with anti-C2 but not anti-Factor B suggested that it was indeed C2. The signal was weak but this may have been due to the non-glycosylated form of the protein (not shown).

1.6 Ligand Blotting of Total Cell Lysates of Schistosoma haematobium Adult Worms and THP-1 Cells

To confirm the interaction between C2 and ShTORE and the human homologue, HuTORE, ligand blotting was carried out. For this, a total protein lysate of THP-1 cells, (and Schistosoma haematobium adult worms, not shown) was run under non-reducing conditions on SDS-PAGE and immunoblotted onto nitrocellulose membrane where it was incubated for 1 h at room temperature with I¹²⁵-labelled C2 (FIG. 10B, lanes 1 and 2) but pre-incubated for 1 h at 4° C. with anti-C2 (lane 2). The C2 has clearly bound to the dimeric (64 kDa) form of TORE and been prevented from doing so by prior incubation of C2 with anti-C2. The same result was obtained with the S. haematobium protein lysate (not shown).

1.7 Inhibition of Classical Pathway-Mediated Lysis of Sheep Erythrocytes by Peptide ed-1

To determine whether the interaction of C2 with ShTORE-ed-1 could affect the normal interaction of C2 with other complement proteins and thereby inhibit complement activation, the effect of the TORE-ed-1 peptide on complement activation was assessed. The ability of the TORE-ed-1 peptide to inhibit the lysis of antibody-sensitized sheep erythrocytes, in the presence of normal human serum, NHS (and therefore in the presence of complement proteins C1 through to C7), in a dose-dependent manner, was used as a measure of its ability to inhibit the Classical Pathway. The extent of haemolysis was determined by spectrophotometric measurement of haemoglobin release at 412 nm. An IC₆₀ value of approximately 10 μM was found (FIG. 11A and B).

As an additional confirmation that TORE-ed-1 specifically binds to human complement C2, C2-deficient human serum with enough C2 added to reconstitute haemolytic activity, was used in the same assay. Preincubation of ShTORE-ed-1 peptide with C2 before addition to C2-depleted serum (Sigma), was found to inhibit haemolytic activity, once more in a dose-dependent manner, (FIG. 11C and D).

The 27 amino acid long TORE-ed-1 synthetic peptide was able to inhibit the haemolytic activity of C2 from the Classical pathway of complement activation at a concentration, 10 μM, that was about 40 times greater than the concentration in NHS. These results suggest that ShTORE and HuTORE bind specifically to human complement C2 and that they inhibit the Classical pathway of complement activation, presumably by inhibiting the formation of the Classical pathway C3 convertase (C4b, 2a).

1.8 Effect of Anti-TORE-ed-1 Antibody on the Susceptibility of Carcinoma Cell Lines to Complement-Mediated Cytotoxicity

Carcinoma cells express complement inhibitors to higher levels than normal cells. It is believed that this overexpression of complement inhibitors may be involved in the. suppression of the in vivo anti-tumour immune response. TORE was found to be overexpressed in Jurkat cells (human T cell line) as compared with normal lymphocytes (not shown).

To ascertain whether TORE is able to protect carcinoma cell lines from complement mediated attack, cells sensitized with antibodies against human whole serum were incubated with increasing concentrations of a TORE-blocking antibody against the putative complement regulator, TORE, before exposure to NHS as a complement source.

A titration was first carried out with increasing concentrations of NHS, FIG. 12a, to find a low enough concentration to be able to easily see any increase in % killing of cells on incubation with increasing amounts of anti-TORE-ed-1. For the experiment, 0.25×10⁶ cells (1×10⁶ cell density) were incubated with 100 μl of anti-human whole serum (1/100) and F(ab′)₂ anti-ShTORE-ed-1 [TBP-1 cells (□) ; U937 cells (Δ)] or normal rabbit IgG as a control (●). After 1 h at 4° C. and two washes in GVB⁺⁺, 100 μl of 10% NHS in GVB⁺⁺ was added for 30 min at 37° C. The percentage killing was determined as the % of cells permeable to trypan blue.

As can be seen in FIG. 12b, there is a significant, four-fold increase, from 10% killing with no anti-TORE-ed-1 antibody to 43% killing with 8 μg/ml anti-TORE-ed-1. This occurred with the macrophage-like THP-1 cells. For the promonocytic U937 cells however, there was no increase in % killing, the level remaining at 10% as in the control, with increasing levels of normal rabbit IgG.

It will be appreciated that overexpression of TORE on carcinoma cell lines provides a means of differentiating carcinoma cells from normal cells, and suggests an approach for cancer therapy by specific targetting of the TORE protein.

1.8.1 In further experiments to ascertain whether CRIT is able to protect carcinoma cell lines from C mediated attack, cells sensitized with antibodies against human lymphocytes (or whole human serum) were incubated with increasing concentrations of an antibody against the putative C regulator, CRIT, before exposure to NHS as a source of C.

A titration was first carried out with increasing concentrations of NHS, FIG. 12A a, to show that lysis was being achieved with this system and to find a convenient serum concentration to be able to see easily any increase in % killing of cells on incubation with increasing amounts of anti-CRIT-ed1.

FIG. 12A b illustrates a significant increase, from 10% killing with no anti-CRIT-ed1 antibody to 43% killing with 10 μg/ml anti-CRIT-ed1 of the macrophage-like carcinoma cell line, TBP-1. With the promonocytic carcinoma cell line, U937, there was a significant increase from 5% killing without anti-CRIT-ed1 antibody to 28% with 12.5 μg/ml. PMA-differentiated U937 promonocytic cells express relatively more CRIT oligomer than other cells examined. IFN-γ-differentiated monocytes also express higher levels of CRIT and this is paralleled by an increase in expression of C2. A significant 3.5-fold increase from 15% killing (with 0 μg/ml anti-CRIT-ed1) to 52.3% (with 10 μg/ml anti-CRIT-ed1) was seen with IFN-γ-differentiated monocytes. Monocytes not treated with IFN-γ showed a non-significant increase in percentage killing from 20% (0 μg/ml anti-CRIT-ed1) to 33.8% (10 μg/ml anti-CRIT-ed1).

The fact that anti-CRIT-ed1 antibody is able to mediate complement-mediated lysis of carcinoma cells point the way to a possible future anti-cancer therapy.

1.9 Effect of TORE Endocytosis on Tyrosine Phosphorylation and Association with Tyrosine Kinase

TORE was immunoprecipitated with anti-TORE-ed-1 and protein A Sepharose. The immunoprecipitate was then immunoblotted with anti-phosphotyrosine. TORE was found to be phosphorylated on tyrosine primarily in the dimeric form. FIG. 13A, lane 5 also shows that TORE was phosphorylated on tyrosine, although to a much lower extent in the trimeric form (96 kDa). A low level phosphorylation in the monomeric (32 kDa) form was also sometimes seen.

The YEDI motif at Tyr¹⁹⁷ of the consensus TORE sequence (FIG. 8) in the cytoplasmic tail of TORE, is an SH2-binding motif with can potentially bind to the SH2 domain of the tyrosine kinases, Syc, SHC or fes. In FIG. 13B, lane 2, a total cell lysate of the THP-1 cell line was immunoprecipitated with anti-ShTORE-ed-1 and immunoblotted with anti-fes. The detection of the 93 kDa fes band confu-ms that TORE can bind fes. As a control a similar co-immunoprecipitation was carried out with a Jurkat cell lysate human T cell line) in lane 1; Jurkat cells do not have fes.

It was also found (FIG. 13C) that neither pre-incubation of THP-1 cells in hypertonic solutions, lanes 1-3 (conditions which inhibit endocytosis) nor the presence of anti-C2, lane 4, nor anti-TORE-ed-1, lane 5, caused a diminution of tyrosine phosphorylation on TORE, compared with cells maintained in normal medium, lane 6, and therefore able to endocytose normally. The same anti-TORE immunoprecipitates used to look at phosphotyrosine levels were then immunoblotted with anti-fes. The association of TORE with fes appeared almost negligible when TORE was prevented from undergoing endocytosis (lanes 1 to 3) compared to the cells in which endocytosis of receptors had been allowed to continue as normal (lane 6). Blocking of TORE with anti-TORE-ed-1 or removing the ligand from the cells by incubation with anti-C2 had the same effect in terms of TORE association with fes and as shown later (4.11), TORE appears to undergo endocytosis which is probably ligand (C2) mediated.

1.10 Homology of ed-1 with C4b

FIG. 14 shows an alignment of the three ed-1 regions sequenced with two regions in C4b, one in the β-chain, the other in the α-chain, these being the parts of C4b which show the greatest homology with TORE-ed-1. The red boxes indicate amino acid identity and the green boxes sequence similarity. Schistosoma and Rat TORE-ed-1 regions show, across their 27 amino acid residues, 35% identity and 42% similarity at the amino acid level with the C4b β-chain, residues 225 to 251 (numbering according to GenBank release p01028) and 27% identity and 46% similarity with C4b α-chain, residues 996 to 1021. FIG. 15A shows a predicted schematic model of how TORE as a (covalently-linked) homo-dimer (the identical TORE molecules are described as TORE-1 and TORE-2 for convenience) in the plasma membrane, might, via its two ed-1 domains, act as a receptor for C2. Although the two ed-1 domains are identical, they are shown within a red and a blue box at the N-terminus of each TORE monomer, to highlight the similarities with the respective regions in the C4b β-chain, presented within a red box, and that in the C4b alpha chain, presented within a blue box. These are shown in FIG. 15B, along with the number of amino acids separating the final Pro within their respective sequences from a disulphide bond linking the α- and β-chains. The amino acids S, E and P which are identically spaced in the TORE-ed-1 domain and the two possible C2-binding domains in C4b, are presented in large red letters. In comparing the ed-1 of TORE-1 (blue box) and the putative C2-binding domain in the C4b α-chain (blue box) amino acid identity is depicted in red letters and similarity in green letters. Amino acids are likewise depicted when comparing the ed-1 of TORE-2 (red box) and the C4b β-chain (red box).

TORE was shown above to form covalently linked oligomers. It is noteworthy that two biological properties of TORE have so far been shown to involve the receptor functioning as a dimer. These include its phosphorylation on tyrosine (FIG. 13A, section 2.9) and the binding of C2 (FIG. 10B, section 2.4). Interestingly, C4b has two regions of similarity to the putative C2-binding ed-1 region of TORE. To see whether there is a precedent for the possibility of C2 binding to two similar regions, be they within C4b or on the two ed-1 regions of a covalently linked TORE, it is also interesting to find that the C2b region of C2, the domain which binds with C4b, consists of three Sushi (SCR) repeats, of 62, 57 and 55 amino acid residues respectively.

It is also interesting to note that the very nature of the disulphide linkage between TORE molecules may bring the ed-1 regions into a closer position for C2 binding. TORE is able to assemble into covalently linked multimers and therefore if the disulphide linkage is between a cysteine residue in the transmembrane (TM) domain of one TORE molecule and the next, this would only be possible between the first TM domain of respective TORE molecules. The first TM domain has 3 cysteines (TM domains 2 and 3 having only 1) and 2 or more cysteines would be required within a TM domain for it to be able to form disulphide bonds to at least two other TM domains.

1.11 TORE Undergoes Endocytosis

The immunofluorescence microscopy studies to localise TORE in the THP-1 cell line showed that only in the presence of endocytosis inhibitors (hypertonic glucose or salt or 2-deoxyglucose) was there an even surface fluorescence of the cells (FIG. 16A) In the absence of endocytosis inhibitors the cells showed a punctate surface fluorescence of the cells (FIG. 16E and F). When cells, similarly pre-incubated, were permeabilised with methanol, they no longer showed the even surface fluorescence in FIG. 16A, but a fluorescence within the nucleus and of the nuclear membrane. However, as Southern blotting indicated the likely existence of subtypes of TORE or other receptors related to TORE, the nuclear fluorescence does not necessarily imply that it is TORE from the plasma membrane being endocytosed and being targeted to the nuclear membrane and nucleus. At this point, the exact target region of the endocytosed TORE is not known.

FIG. 17A and B shows immunoelectron micrographs in which the receptor appears to cluster (indicated by arrow) in what are probably clathrin-coated pits (clathrin-coated, because endocytosis was inhibited in hypertonic solution) on the plasma membrane of THP-1 cells and to then undergo endocytosis into the cell FIG. 17, C.

Although C2 by itself has no deleterious effects and no C2 anaphylatoxin has been reported, a negative feedback mechanism may be postulated, whereby on interaction of TORE with C2, TORE becomes internalized, thereby removing C2 and preventing the formation of the C3 convertase of the Classical pathway. As described above, C2-induced TORE internalization was inhibited by treatment of the cells with various inhibitors of clathrin coated pit-mediated endocytosis. The resulting decrease in ligand-dependent receptor internalization produced a corresponding decrease in the level of association of TORE with fes, a cytoplasmic tyrosine kinase, which is able to translocate to the nucleus and which is believed to be involved in the terminal differentiation of monocytes and neutrophils.

### Reference Example 2

R2.1 Cloning of DNA Encoding the SmTORE Protein

ShTORE was cloned by screening a S. haematobium adult worm cDNA library in λgt11 using vaccinated baboon serum (VBabS) according to standard methodology (11). Specifically, the S. mansoni homologue SmTORE was cloned by screening an S. mansoni adult worm cDNA library using the full length ShTORE cDNA as a probe in a standard procedure (3). For sequencing, ShTORE and SmTORE were subeloned into pUC18. The nucleotide sequence for SmTORE open reading frame (ORF) is presented in FIG. 18, along with the translation into the protein sequence.

R2.2 Antibodies to SmTORE Cross React with the ShTORE Protein

A rabbit polyclonal antibody was raised against a peptide based upon the N-terminal 27 residues of the SmTORE sequence using standard procedures. FIG. 19A, lane 1 shows recognition of the predicted 31.2 kd SmTORE protein in a Western blot of an adult worm lysate. The slightly larger ShTORE protein was also recognised by the same polyclonal antibody (FIG. 19A, lane 3).

Figures

FIG. 1(A) Nucelotide and deduced amino acid sequence of ShTORE. All ATG sequences in the 5′ region of the sequence are numbered in superscript. The 2 putative translation start codons are ATG⁴ and ATG⁶. The stop codons are also numbered in superscript. Part of the vector sequence, including the EcoRI site (underlined) 3′ of the TORE ORF is indicated in lowercase. (B) Kyte and Doolittle plot of ShTORE showing predicted hydrophobic transmembrane and hydrophilic domains.

| | |
|---|---|
| 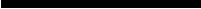 | primers for sequencing and PCR |
| 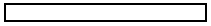 | transmembrane domain |
| 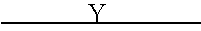 | tyrosine sulfation site |
| 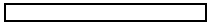 | amphipathic helix |
| 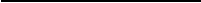 | casein kinase II phosphorylation site |
| 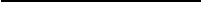 | protein kinase C phosphorylation site |
| 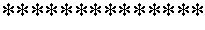 | myristoylation site |

FIG. 2(A) Predicted membrane topology of ShTORE (i) shows the 32 kDa TORE molecule starting at ATG⁴ and (ii) the predicted 27 kDa molecule from ATG⁶. (B) Helical wheel representation of ShTORE-id2, Lys¹⁹⁶-Ala²⁰⁸. The hydrophobic amino acid residues are boxed.

FIG. 3(A) Recognition of native ShTORE in adult worm preparation of S. haematobium by rabbit anti-ShTORE-ed-1 antibodies in lanes 1 and by rabbit pre-bleed in lane 2. (B) Western recognition of ShTORE expressed in L cells (exposed to tunicamycin), lane 1 and in L cells (without exposure to tunicamycin), lane 2.

FIG. 4(A) High stringency Southern blot of S. haematobium genomic DNA digested with: lane 1, EcoRI; lane 2, PstI; lane 3, HindIII, probed with full length ShTORE cDNA and (B) the same blot probed under low stringency conditions. (C) High stringency Southern blot of T. cruzi genomic DNA cut with HindIII, lane 1 and uncut, lane 2, probed with full length ShTORE cDNA (D) Northern blot (ethidium bromide gel below) of total RNA from different stages of C. elegans and S. haematobium probed with fall length ShTORE cDNA; stages of C. elegans: lane 1, C1; lane 2, C2; lane 3, C3; lane 4, C4; lane 5, adult worm. Stages of S. haematobium: lane 5, adult worm; lane 6, schistosomular.

FIG. 5 Immunoelectron microscopy of surface tegument (T) region of S. Haematobium adult worms [showing also surrounding host plasma (PL)] probed with anti-ShTORE-ed-1 [(C), x7,500; Bar is 1 μm], [(E), x7,500; Bar is 500 μm] and [(F), x20,000; Bar is 200 nm] and antibody pre-absorbed with ShTORE-ed-1 peptide [(A), x300; Bar is 500 μm] and [(B), x7,500; Bar is 1 μm] and pre-immune serum [(D), x7,500; Bar is 500 nm] to reveal location of ShTORE: Label is confined to the tegumental outer membrane, in contact with the host plasma and the surface pits (P) and the interconnecting channels into which they branch. The longitudinal muscle (LM) and transverse muscle (TM) fibres are devoid of label.

FIG. 6 Southern blot of human and Schistosoma mansoni genomic DNA probed with SmTORE full length EDNA under low stringency conditions. Human genomic DNA is digested with: lane 1, EcoRI; lane 2, PstI; lane 3, HindIII. Schistosoma mansoizi genomic DNA digested with: lane 1, EcoRI; lane 2, PstI; lane 3, HindIII

FIG. 7 Schematic of ShTORE showing membrane topology and important sequence motifs including a myristoylation site, a YXXI endocytosis motif followed by a amphipathic helix, a serine/threonine rich region and different consensus phosphorylation sites for various tyrosine kinases, all in the cytoplasmic tail. Extracellular domains 1 (ed1) and 2 (ed2) are indicated, as well as the intracellular domains 1 (id1) and 2 (id2).

FIG. 8 Amino acid sequence alignment (CLUSTALW) of TORE from Rat (RaTORE), Schistosoma mansoni (SmTORE) and Schistosoma haematobium (ShTORE). Amino acid identity is indicated below the alignment by an asterisk and similarity by dots. The three transmembrane domains, TM, are indicated by bars above the sequences.

FIG. 9 Western blotting using anti-ShTORE-ed1 to indicate cellular distribution of TORE. (A) Lanes 1 and 3, total cell lysate of Jurkat cells under non-reducing conditions. Lane 2 and 4, total protein lysate of human platelets. (B) Total protein extracts of various human haematopoietic cell lines run under reducing conditions: Lane 1, Jurkat; lane 2, Raji; lane 3, TBP-1; lane 4, U937+PMA; lane 5, U937−PMA; lane 6, ECV-304; lane 6, S. haematobium. (C) TBP-1 cell lysate run under non-reducing conditions (lane 1) and reducing conditions (lane 2).

FIG. 10 Ligand identification by receptor affinity chromatography using the ed1 putative ligand binding domain. Lane 1, aliquot of one fraction of the low pH elution from a chromatography carries out on 1L of conditioned, serum-free medium which had maintained THP-1 cells. Lane 2 is a pool from several fractions of the same chromatography carried out on 1L of serum-free medium which had not maintained any cells. (B) Ligand blotting showing binding of human complement protein C2 to the dimeric form of HuTORE. A total cell lysate of the human THP-1 cell line, separated by SDS-PAGE under reducing conditions and transferred onto nitrocellulose membrane was probed with radiolabelled C2, lane 1. The same lysate was probed in lane 2 but as a control, the C2 radiolabelled probe was preincubated with anti-C2 before exposure to the blot.

FIG. 11(A) and (B) Effect of increasing TORE-ed1 peptide on the complement-mediated lysis of sensitized sheep red blood cells in the presence of normal human serum as a source of complement. FIG. 11(C) and (D) Effect of increasing TORE-ed1 peptide preincubated with C2 on haemolysis in the presence of human serum in which C2 is limiting. FIG. 11(E) effect of various Tore peptides and Tore related C4b-derived peptides on complement mediated lysis.

FIG. 12 Blocking the putative complement regulator receptor TORE, which is over-expressed on the surface of carcinoma cell lines, with anti-TORE-ed1 and its effect on the complement-mediated killing of normal human serum (NHS) as determined by typan blue uptake (A) Mean effect of increasing NHS on the % killing of antibody-sensitized human TBP-1 and U937 carcinoma cells after 37° C. incubation for 30 min. (B) Effect of anti-TORE-ed1 F(ab′)₂ on the percentage killing of antibody-sensitized THP-1 cells ( [symbol] ) and U937 cells (Δ) in the presence of NHS (10%) after 37° C. incubation for 30 min. As a control, the mean effect on U937 and THP-1 cells of increasing normal rabbit IgG, in the same assay was also detected.

FIG. 12A. Effect of anti-CRIT-ed1 on complement-mediated toxicity of carcinoma cell lines. Cells (0.25×10⁶) were incubated with 100 μl of anti-human lymphocyte serum (1/30) or rabbit prebleed/normal rabbit IgG as a control followed by incubation with anti-CRIT-ed1. After 1 h at 4° C. and two washes in GVB⁺⁺, 100 μl of 10% NHS in GVB⁺⁺ was added for 30 min at 37° C. The percentage killing was determined as the % of cells permeable to trypan blue. (a)-(d), blocking the putative C regulatory receptor CRIT, which is over-expressed on the surface of carcinoma cell lines, with anti-CRIT-ed1 and its effect on C-mediated killing by NHS, as determined by trypan blue uptake. (a) Mean effect of increasing NHS on the % killing of antibody-sensitized (using anti-lymphocyte serum) human THP-1 and U937 carcinoma cells after 37° C. incubation for 30 min. Effect of increasing anti-CRIT-ed1 on the % killing in the presence of 10% NHS, after 37° C. incubation for 30 min, of antibody-sensitized (b) THP-1 cells, (c) U937 cells and (d) IFN-γ-differentiated monocytes, versus untreated monocytes. As a control, in (b) and (c) the mean effect of increasing anti-CRIT-ed1 on THP-1 and U937 cells, treated with normal rabbit prebleed or rabbit IgG, was also detected in the same assay.

FIG. 13(A) Tyrosine phosphorylation of TORE on THP-1 cells and Schistosoma haematobium. Lane 1 Anti-phosphotyrosine immunoblot (IB) of anti-TORE-ed1 immunoprecipitate (IP) of THP-1 cell lysate (and S. haematobium in lane 3), on protein A Sepharose; lane 2 no anti-TORE-ed1 control; lane 4; no anti-phosphotyrosine control; lane 5; twice the protein loading in lane 1. (B) Co-immunoprecipitation of Jurkat cells (lane 1) and THP-1 (lane 2). Both lanes represent anti-fes IBs of anti-TORE-ed1 IPs. (C) Effect on THP-1 cells of preincubation with endocytosis inhibitors, and antibodies that block C2 binding, on the levels of TORE phosphorylation on tyrosine and of association with the fes tyrosine kinase. Cells were preincubated at 37° C. for 20 min. as follows: Lane 1, 0.06% NaN₃ and 10 mM 2-deoxyglucose; lane 2, 225 mM NaCl; lane 3, 0.45M sucrose; lane 4, anti-C2; lane 5, 5 μg/ml anti-TORE-ed1; lane 6, blank control.

FIG. 14 Amino acid sequence alignment of the N-terminal extracellular domains (ed1) of ShTORE, SmTORE and RaTORE with two regions from the α- and β-chains of human C4b with which they show homology. Residues coloured red are identical and those coloured green are similar. A consensus sequence is also shown.

FIG. 15(A) Schematic diagram showing TORE homo-dimer (represented as identical TORE-1 and TORE-2) binding to C2. The ed1 domains have been highlighted in a red or blue box to show the homology (B) with two different regions also indicated by a red and blue box in the β-chain and β-chains respectively of C4b.

FIG. 16 Immunofluorescence microscopy of THP-1 cells to indicate endocytosis of TORE. Panel C, Phase contrast of cells and panel A, surface fluorescence of same cells incubated in the presence of hypertonic salt solution (identical fluorescence seen using other inhibitors of clathrin-coated pit-mediated endocytosis). In the absence of endocytosis inhibitors there was only a punctuate surface fluorescence (panel E and F) but when such cells were permeabilised with methanol as shown in Panel B (equivalent phase contrast in panel D) there was a preponderance of fluorescence in the nucleus and nuclear membrane.

FIG. 17 Immunoelecron microscopy of the surface of THP-1 cells (A to C) using anti-TORE-ed1 antibody and anti-rabbit gold labelled conjugate to indicate likely endocytosis of TORE via clathrin-coated pits (indicated by arrows). For panel D the anti-TORE-ed1 antibody was pre-absorbed with TORE-ed1 peptide. The Bars are 500 nm (panels A and C), 250 nm (panel B) and 1 μm (panel D).

FIG. 18 S. mansoni DNA sequence and protein sequence, along with a comparison of the S. mansoni and S. haematobium protein sequences.

FIG. 19(A) Recognition of native SmTORE/ShTORE from total S. mansoni/S. haematobium protein lysates (lanes 1 and 3, respectively) by antibody raised against a peptide based on the predicted extracellular N-termius of SmTORE. The respective same protein lysates were probed with pre-immune serum in lanes 2 and 4. (B) Various recombinant antigens probed with VBabS (lanes 1-3 and 7-8) and NBabS (lanes 4-6). Lanes 1 and 4; ShTORE cloned in reverse orientation. Lanes 2 and 5; ShTORE-GST. Lanes 3 and 6; ShTORE cleaved (with thrombin) and purified from GST. Lane 7; ShTORE-id2 cloned in reverse orientation. Lane 8; ShTORE-id2-GST. Lane 9; ShTORE-id2 cleaved (with thrombin) and purified from GST.

FIG. 20 Nucleotide sequence and translation of RaTORE obtained by PCR using degenerate primers. The primers were designed based upon the Schistosoma sequence and Rattus codon usage.

FIG. 21; shows amino acid identity between ed-1, ed-2 and C4b.

FIG. 22: Mice (7 or 8 per group) injected with human albumin (HA) 10 mg/kg i.v. (200 μl of a solution 1 mg/ml in Evans blue1%). After 5 min anti-HA injected s.c. in situ (in ear). Test compounds then injected and after 4 h evaluations made for reduction of inflammation. Oedema was evaluated by weight. Plasma extravasation as measure of microvascular permeability or haemorrage was measured by extraction of extravasated Evan's blue dye with formamide from injected ears. As well as subcutaneous (s.c.) administration of H17 (the test compound of formula HEVKIKHFSPY at two concentrations), it was also given intravenously (0.1 mg/kg). The positive control for reduction in inflammation in this experiment was cromolyn. As can be seen H17 (at a 1 μg dose) gave a 30% reduction of haemorrage and 28% reduction of oedema (both comparable with the positive control).

1 Use of a peptide comprising the sequence of SEQ ID NO 1 or SEQ ID NO 2, or fragment thereof, or comprising a mutant or variant of SEQ ID NO 1, SEQ ID NO 2 or fragment thereof, in medicine.

2 Use of a peptide according to claim 1 in the preparation of a medicament for modulation of complement activity.

3 Use according to claim 2 wherein the medicament is for inhibition of complement activity.

4 Use according to any preceding claim, wherein the peptide is capable of interaction with human complement protein C2.

5 use according to any preceding claim, wherein the peptide is capable of inhibiting the interaction of human complement protein C2 with human complement protein C4b:

6 Use according to any preceding claim, wherein the peptide comprises the consensus sequence EVKI-Xn-PY, wherein X is any amino acid and n=1-6.

7 Use according to any preceding claim, wherein the peptide is a dimer or higher oligomer.

8 Use according to claim 7 wherein the peptide comprises the general sequence E-V-K-X(0,8)-E-V-K-I-X(4)-P-Y

9 Use according to any preceding claim, wherein the peptide is a cyclic peptide.

10 Use according to any preceding claim, wherein the peptide is derived from human C4b complement protein.

11 A peptide according to any preceding claim suitable for use in the present invention, wherein the peptide is not full length SmTORE or full length ShTORE.

12 A peptide according to claim 11, comprising the sequence of SEQ ID NO 1 or SEQ ID NO 2, or fragment thereof, or mutant or variant thereof.

13 A peptide according to any of claims 11-12, wherein the peptide is 11 amino acids or shorter in length

14 A peptide according to claim 11-13 comprising the consensus sequence EVKI-X(n)-PY.

15 A peptide according to claim 14 selected from the peptides: MSPSLVSHTQKNERGSHEVKIEHFTPY; MSPSLVSYTQKNERGSHEVKIKHFSPY; MSPSLVSDTQKHERGSHEVKIKHFSPY; FEVKITPGKPY; HEVKIKHFSPY; CHEVKIKHFSPYIAV, or FEVKKYVLPNFEVKITPGKPY.

16 A peptide according to claim 11 or 12, comprising the general sequence X-S-X-S-D-X(0,1)-R-X(3)-H-X(2)-T-X(0,1)-G-P, where X(0,1) is none or any amino acid.

17 A peptide according to claim 16 comprising the sequence SSTSDIRLVIHTKTGPIYIK, TSLSDRYVSHEFETEGP, SSTSDLRLMIHTKTGPIYIK or SSTSDLRLMIHTKTGPIYIK.

18 A pharmaceutical composition comprising a peptide according to any of claims 11-17 in combination with a pharmaceutically acceptable carrier.

19 A pharmaceutical composition according to claim 18 in a form suitable for oral delivery.

20 A pharmaceutical composition according to claim 18 or 19, wherein the peptide is in combination with liposomes.

21 A polynucleotide encoding a peptide according to any of claims 11-17.

22 A polynucleotide capable of hybridising to a polynucleotide according to claim 21.

23 A polynucleotide according to claim 22, comprising antisense RNA.

24 A vector comprising a polynucleotide according to any of claims 21-23.

25 A cell comprising a vector according to claim 24.

26 An antibody specific to a peptide according to any of claims 11-17.

27 A method of treatment comprising administering an effective amount of a peptide or pharmaceutical composition according to any of claims 11-20 to a patient in need of such treatment, wherein the patient is affected by a disease having a component of complement disorder.

28 A method according to claim 27, wherein the disease is selected from an autoimmune disease such as rheumatoid arthritis, multiple sclerosis, systemic lupus erythematosus, myasthenia gravis, neuromuscular diseases such as dermatomyositis [DM], Lambert-Eaton mysthenic syndrome [LEMS]or following cardiopulmonary bypass surgery; myocardial infarction or Alzheimer's disease.

29 A method of treatment of a patient having a xenotransplant or is undergoing xenotransplantation comprising administering an effective amount of a peptide or pharmaceutical composition according to any of claims 11-18.

30 A method of inhibiting complement activation in a patient, comprising administering an effective amount of a peptide or pharmaceutical composition according to any of claims 11-20.

31 A method of treatment of a patient affected by a complement disorder comprising administering an effective amount of an antibody according to claim 26, in order to modulate complement activity.

32 A method of treatment for a patient with cancer, comprising administering an effective amount of a peptide, pharmaceutical composition, nucleic acid or antibody according to any of claims 11-21 and 24.

33 Use of a peptide, pharmaceutical composition, polynucleotide or antibody according to any of claims 11-24 in the preparation of a vaccine against schistosomal, trypanosomal or malarial infection or disease.

34 A vaccine according to claim 33.

35 Use of a peptide according to any of claims 11-18 or antibody according to claim 26 detection of pathogens.


SEQUENCE LISTING (47 sequences)

SEQ ID NO: 1 - protein, 27 aa, shistosoma haematobium
MSPSLVSDTQKHERGSHEVKIKHFSPY

SEQ ID NO: 2 - protein, 22 aa, shistosoma haematobium
SSTSDIRLVIHTKTGPIYIKST

SEQ ID NO: 3 - protein, 26 aa, shistosome haematobium
SPSLVSDTQKHERGSHEVKIKHFSPY

SEQ ID NO: 4 - protein, 26 aa, shistosome mansoni
SPSLVSYTQKNERGSHEVKIKHFSPY

SEQ ID NO: 5 - protein, 26 aa, rattus rattus
SPSLVSHTQKNERGSHEVKIEHITPY

SEQ ID NO: 6 - protein, 16 aa, shistosome haematobium
SSTSDIRLVIHTKTGP

SEQ ID NO: 7 - protein, 20 aa, shistosoma mansoni or rattus rattus
SSTSDLRLMIHTKTGPIYIK

SEQ ID NO: 8 - protein, 27 aa, rattus rattus
MSPSLVSHTQKNERGSHEVKIEHFTPY

SEQ ID NO: 9 - protein, 286 aa, Schistosome haematobium
MSPSLVSDTQKHERGSHEVKIKHFSPYIAVCVTTFSLAFCCFMVHAAITRQPTHLLPFFFIQVFDLIICLIHILGFMSSTSDIRLVIHTKTGPIYIKSTGLTFIILSISCMMLAFKAYCLGMVWDCYKYLMLNRRGNLLDDWYSDQWGHLSTFWSLLRTGRNRGNNSIGNSGSPNEPNTRPRPDTITYDPANDLPKYEDILKIRNAYAPPPYYCSNTNGNVNTTTTDAVTTNTTITSATTANATTTITTNANTNTSTTTSVISPLTTTNKDDTQINNASSNAHSSC

SEQ ID NO: 10 - protein, 281 aa, Shistosome mansoni
MSPSLVSYTQKNERGSHEVKIKHFSPYIAVCVTTFSLAFCCFMVHGAITKQPTHLLPFFFIQVFDLIICLIHILGFMSSTSDLRLMIHTKTGPIYIKSTGFTFIILSISCMMLAFKAYRLGTVWDCYKYLMLNRKSNLLDDWYSDQWGHLSTFWSLLRAGPNGSNNPNGNSGSRNEPNTRLRPEPITYGPANDLPKYEDILKIPTNAYAPPPYYCSNINGNVNTTEASAVTTNTSNSATAANTTTTTTNTGTTTSVISTVTTINKDDTQINSAPSNAHSSC

SEQ ID NO: 11 - protein, 282 aa, rattus rattus
MSPSLVSHTQKNERGSHEVKIEHFTPYIAVCVTTFSLAFCCFMVHGAITKQVNPLTSRCPLIQVFDLIICLIHILGFMSSTSDLRLMIHTKTGPIYIKSTGLTFIISSISCMMLAFKPYRLGTVWDCYKYLMLNPRGILLDDWYPDQCGPLSHFSASLLPAGRNWGNIGGGMWSLNEHNRRLRPEPVTYGPANDVPKYEDILKIRTNAYVLPPYYCSNINGNDNTTEGSAVTTNTSNCGTAANSTTSTTNTGSTTSVISTVTTINKDDTQINSAPWNAHSSC

SEQ ID NO: 12 - DNA, 630 bp, Shistosome haematobium
ccacaatgtgagtctgaaaccaattttcattatgatattccaccaggatataaagacgatgtacttgtcgacgtcaataatatgtctccaagtctagtgtcggatactcagaaacatgaacgtggatcccacgaagtcaaaataaaacatttcagtccttacattgctgtttgcgtgacaactttctctttggcgttttgttgcttcatggtccatgcagcaatcacaagacagccaactcacttactcccgtttttctttattcaagtctttgatcttatcatatgtttaattcacatactcggattcatgtcctccacatcagatatacgcttggtaattcacacaaaaacagggcctatttacatcaaatctacgggtttgacttttatcatattgtccatctcatgcatgatgttggcattcaaagcctattgtcttggtatggtatgggactgttataaatacttaatgctaaatcgaagaggcaacctacttgatgattggtattccgaccagtggggtcatttgtcaactttttggagtttacttcggactggtcgtaatcgaggcaataattcgattgggaactctggttctcctaatgagcctaatacaagacctcgtcct

SEQ ID NO: 13 - DNA, 846 bp, shistosome mansoni
  misc_feature (621)..(621): N is any nucleotide
atgtctccaagtctagtgtcttatactcagaaaaatgaacgtggatcccatgaagttaaaataaagcatttcagtccttacattgctgtttgtgtgacaactttctctttggcgttttgctgcttcatggtccatggagcaatcacaaaacagccaacccacttactcccgtttttctttattcaagtatttgatcttatcatatgtttaattcacatactcggattcatgtcctccacatcagatctacgcttgatgattcacacaaaaacggggcctatttacatcaaatctacaggtttcacttttatcatattgtccatctcatgcatgatgttggctttcaaagcctatcgtcttggtacggtatgggactgttataaatacttaatgctaaatcgaaaaagcaacctacttgatgattggtattccgaccagtggggtcatttgtctactttttggagtttacttcgggctggtcccaatggaagcaacaatcccaatgggaactctggttctcgtaatgagcctaacacgagacttcgtcctgaaccaattacatacggtccagctaacgatctaccaaagtatgaggatatattgaaaattccgacaaatgcntacgctcctccaccttattactgttccaacatcaacggaaatgtcaatacaactgaagctagtgctgttactaccaatactagtaattctgctactgcggctaatactactactactactactaatactggtactacaactagtgtgatatcaacagttacaacaattaacaaggatgatacccaaatcaatagtgcaccatcgaatgctcactcttcttgctaa

SEQ ID NO: 14 - DNA, 849 bp, rattus rattus
atgtctccaagtctagtgtctcatactcagaaaaatgaacgtggatcccatgaagttaaaatagagcatttcaccccctacatcgctgtttgtgtgacaactttctctttggcgttttgctgcttcatggtccatggagcaatcacaaaacaggtcaacccacttacttctcgttgtcctcttatccaagtatttgatcttatcatatgtttaattcacatactcggattcatgtcctccacatcggatctacgcttgatgattcacacaaaaacggggcctatttacatcaaatctacaggtctcacttttatcatatcgtccatctcatgcatgatgttggctttcaaaccctatcgtcttggtacggtatgggactgttataaatacttaatgctaaatccacgaggcatactacttgatgattggtatcccgaccagtgcggtcctctgtctcacttttctgcgagtctacttccggctggtcgcaattggggcaacattggagggggaatgtggtctcttaatgagcataacaggagacttcgtcctgaaccagtaacatacggtccagctaacgatgtaccaaagtatgaggatatattgaaaattaggacaaatgcgtacgttcttccaccttattactgttccaacatcaacggaaatgacaatacaactgaaggtagtgctgttactaccaatactagtaattgtggtactgcggctaatagtactactagtactactaatactggcagtacaactagtgtgatatcaacagttacaacaattaacaaggatgatacccaaatcaatagtgcaccatggaatgctcactcttcttgctaa

SEQ ID NO: 15 - protein, 26 aa, Homo sapiens
SPGGVASLLRLPRGCGEQTMIYLAPT

SEQ ID NO: 16 - protein, 27 aa, homo sapiens
SNSSTQFEVKKYVLPNFEVKITPGKPY

SEQ ID NO: 17 - protein, 21 aa, homo sapiens
FEVKKYVLPNFEVKITPGKPY

SEQ ID NO: 18 - protein, 22 aa, artificial sequence
  Description of artificial sequence contiguous C-terminal positions of ed-1 peptide
HEVKIKHFSPYHEVKIKHFSPY

SEQ ID NO: 19 - protein, 16 aa, homo sapiens
TSLSDRYVSHFETEGP

SEQ ID NO: 20 - protein, 27 aa, shistosoma mansoni
MSPSLVSYTQKNERGSHEVKIKHFSPY

SEQ ID NO: 21 - protein, 26 aa, artificial sequence
  Description of artificial sequence sh-TORE-ed1 (H 17)cyc1
CHEVKIKHFSPYCCHEVKIKHFSPYC

SEQ ID NO: 22 - protein, 26 aa, artificial sequence
  Description of artificial sequence sh-TORE-ed1 (H 17)cyc2
CHEVKIKHFSPYCCYPSFHKIKVEHC

SEQ ID NO: 23 - protein, 11 aa, shistosoma haematobium
HEVKIKHFSPY

SEQ ID NO: 24 - protein, 11 aa, homo sapiens
FEVKITPGKPY

SEQ ID NO: 25 - DNA, 24 bp, artificial sequence
  Description of artificial sequence primer
cgcgatgtcyccnasactagtatc

SEQ ID NO: 26 - DNA, 25 bp, artificial sequence
  Description of artificial sequence primer
cgcgttarcaagaagartgngcrtt

SEQ ID NO: 27 - protein, 27 aa, Artificial sequence
  Description of artificial sequence consensus sequence
MSPSLVSXTQKXERGSHEVKIXHFXPY

SEQ ID NO: 28 - protein, 7 aa, Shistosoma haematobium
MSPSLVS

SEQ ID NO: 29 - protein, 9 aa, Shistosoma haematobium
ERGSHEVKI

SEQ ID NO: 30 - protein, 7 aa, Artificial sequence
  Description of artificial sequence consensus sequence
EVKIXPY

SEQ ID NO: 31 - protein, 7 aa, Artificial sequence
  Description of artificial sequence consensus sequence
EVKIXPY

SEQ ID NO: 32 - protein, 4 aa, Shistosoma haematobium
EVKI

SEQ ID NO: 33 - protein, 5 aa, Shistosoma haematobium
HEVKI

SEQ ID NO: 34 - protein, 16 aa, Artificial sequence
  Description of artificial sequence consensus sequence
XEVKXXPXXEVKXXPX

SEQ ID NO: 35 - protein, 11 aa, Artificial sequence
  Description of artificial sequence consensus sequence
EVKXEVKIXPY

SEQ ID NO: 36 - protein, 13 aa, Artificial sequence
  Description of artificial sequence consensus sequence
SXSXKXEVKIXPY

SEQ ID NO: 37 - protein, 10 aa, Artificial sequence
  Description of artificial sequence consensus sequence
SXVXRGXEXP

SEQ ID NO: 38 - protein, 5 aa, Artificial sequence
  Description of artificial sequence consensus sequence
SXEXP

SEQ ID NO: 39 - protein, 20 aa, Shistosoma haematobium
SSTSDIRLVIHTKTGPIYIK

SEQ ID NO: 40 - protein, 17 aa, Artificial sequence
  Description of artificial sequence consensus sequence
XSXSDXRXXXHXXTXGP

SEQ ID NO: 41 - protein, 14 aa, Artificial sequence
  Description of artificial sequence consensus sequence
XSXSDXRXHXTXGP

SEQ ID NO: 42 - protein, 26 aa, Artificial sequence
  Description of artificial sequence consensus sequence
HILGFMSSTSDXRLXIHTKTGPIYIK

SEQ ID NO: 43 - protein, 22 aa, Artificial sequence
  Description of artificial sequence consensus sequence
SSTSDXRLXIHTKTGPIYIKST

SEQ ID NO: 44 - protein, 6 aa, Shistosoma haematobium
PKYEDI

SEQ ID NO: 45 - protein, 15 aa, Shistosoma haematobium
CHEVKIKHFSPYIAV

SEQ ID NO: 46 - DNA, 1220 bp, Shistosoma haematobium
  CDS (82)..(942)
ccacaatgtgagtctgaaaccaattttcattatgatattccaccaggatataaagacgatgtacttgtcgacgtcaataatatgtctccaagtctagtgtcggatactcagaaacatgaacgtggatcccacgaagtcaaaataaaacatttcagtccttacattgctgtttgcgtgacaactttctctttggcgttttgttgcttcatggtccatgcagcaatcacaagacagccaactcacttactcccgtttttctttattcaagtctttgatcttatcatatgtttaattcacatactcggattcatgtcctccacatcagatatacgcttggtaattcacacaaaaacagggcctatttacatcaaatctacgggtttgacttttatcatattgtccatctcatgcatgatgttggcattcaaagcctattgtcttggtatggtatgggactgttataaatacttaatgctaaatcgaagaggcaacctacttgatgattggtattccgaccagtggggtcatttgtcaactttttggagtttacttcggactggtcgtaatcgaggcaataattcgattgggaactctggttctcctaatgagcctaatacaagacctcgtcctgatacaattacatacgatccagctaacgatctaccaaagtatgaggatatcttgaaaattcgaaatgcctacgctcctccaccctattactgttctaacaccaacggaaatgtcaatacaactacaactgatgctgttactaccaatacgactattacttctgctactacggctaatgctactactactattactactaatgctaatactaatactagtactacaactagtgtgatatcaccacttacaacaactaacaaggatgatacccaaatcaataatgcatcatcgaatgctcactcttcttgttaatctgatttaattgttaactacatacgcactgattctaaacattacaaaaacgaaacattctaaaagtatagttttttttacgtcaaatcacgataaatgatttttggcttattagaaaaactcgtgtgtttatgtttacacacttattttcattttcattttattcattacacctgtttgtttactttgttcattgtttctcttcactaaagttgatctgtggaaattctaaatgttatttctttaccaaaaaaaaaaggaattccgtccgatactga

SEQ ID NO: 47 - protein, 286 aa, Shistosoma haematobium; identical to SEQ ID NO: 9